SEQUENCE LISTING (38 sequences)

SEQ ID NO: 1 - DNA, 966 bp, synthetic construct
  source 1..966: mol_type = other DNA
atggcagcttcaggagaaccccagaggcaatggcaagaggaagttgctgctgtggtggtcgtcggctcctgcatgactgatctcgtgagcttgacctctcgcctgccaaagacgggtgagacaattcacggccataaattcttcattggttttggaggtaagggtgccaaccagtgtgttcaagcggcgcgtctgggtgctatgaccagcatggtatgcaaagtgggcaaggacagcttcggcaatgactacattgaaaacttgaagcaaaacgatatttccaccgagttcacctatcagaccaaagacgccgctactggcaccgcaagcattattgtgaacaacgaaggtcagaacattatcgtgatcgtggcgggtgcgaatctgctgctcaacaccgaggacctgcgcgcagcggcgaacgtgatctctcgtgcaaaagtcatggtgtgccaactggaaatcaccccagcgaccagcctggaagctctcacgatggcacgccgttcaggtgttaagaccttgtttaacccggctccggccattgcggacctggacccgcagttttacaccctgtccgatgttttttgttgtaatgagagcgaagccgagatcctgaccggcctgacggttggcagtgcagcggacgcgggcgaagcagccctggttctgctgaaacgtggttgccaggttgttatcatcacccttggcgcggagggctgcgtggtcctgagccagactgagccggaaccgaagcacatcccgaccgagaaagtgaaggcggttgataccaccggtgcgggtgattcgttcgtgggtgcgctggcgttttacctggcgtattacccgaatttgtctttggaggacatgctgaatcgttccaacttcatcgccgcggttagcgttcaagcggcgggtacgcagagcagctatccgtataaaaaagacttaccgctgacgctgttc

SEQ ID NO: 2 - DNA, 951 bp, synthetic construct
  source 1..951: mol_type = other DNA
atgagtaacgaatatggcgataaaaatttgaagattttctctttgaacagtaaccccgaactggcaaaggagattgccgacaacgttggcgtacagcttggtaaatgttccgtgacgcgcttcagtgatggtgaggtacaaatcaacatcgaagaatcaatccgtggatgcgactgttacattattcaatcaacttcagcacctgtcaatgaacacatcatggagttgttaattatggttgatgcgttgaagcgtgcatctgctaagactatcaacattgttattccatactacggatatgcccgtcaggatcgcaaggcacgcagccgcgaacccattactgccaagttatttgcaaacttgttggaaaccgccggtgccacgcgtgtgatcgctttggatattcacgctccgcagattcaggggttcttcgatatcccgatcgaccatctgatgggcgtcccaatcctgggtcattattttgaaggcaaggatttaaaagacatcgtcattgtgtctcctgatcacgggggagtcacgcgcgctcgtaaacttgcagaccgcttgaaagcaccaattgccatcattgacaagcgtcgtccccgccccaacgaagtggaggtcatgaatatcgtcggaaatgtagagggaaagaccgccatccttattgatgacatcattgatacagcgggcacaatcacattggctgctaacgcgcttgtggagaatggagcagcggaggtttacgcgtgctgcactcatccggtactgtccggtccagcggtggagcgcatcaataattctaagatcaaggaattagttgttacaaactcaatcaagttgccagaggagaagaaaattgaacgtttcaagcaattgagtgtagggcccttactggcagaagccatcatccgcgtgcacgaaaagcagagtgtctcgtacctgttttcg

SEQ ID NO: 3 - DNA, 564 bp, synthetic construct
  source 1..564: mol_type = other DNA
atgtcagatgacaggataaattatctaaaaaacaagctggtgcagtacccggattttccgaaaaaaggtatcctgttcgaagacatcatgccgatttttcaggacccgagagcattcggcattctgatcgacctgctgcttgaggccgttgaaaccgagttcaacaacatcgatgttattgtgggtctggaggcgcgtggttttctgttcggccctacgttggcgctgcgcgcgaactgcgcgtttgttccggttcgtaagccgaataagctgccgggtgatctggtggtggtttcctacaataaagagtatagcaccgacagcttcgcgattcaaaaaggtacgatcaaaccaggccagcgtgttctgattgtcgatgacatccttgccaccggcggtacggctctggcggcagatgagctggtcacccgtctgggtggtgaattagtgggccacttgttcttgttggagttaacctttctgcaaggtcgtaagcgcttgatggcaccgacctataccttgctgactggccaagacgaggctccggatggctctgagttcgcg

SEQ ID NO: 4 - DNA, 447 bp, synthetic construct
  source 1..447: mol_type = other DNA
atgtcatttaggttcggacaacacctaataaagccgagcgttgtctttctgaaaaccgaactgagcttcgccttggttaaccgcaagccggttgttccgggtcatgtgctggtgtgcccgctgagacctgttgaacgttttcgcgacatgagcccggaggaggttgcagacttgtttcaagcggcgcagcgcgtgggcacggtggtcgagaagcacttccaaggtacttcgctgaccttttccatgcaggatggtccagaggccggccagaccgtaaaacacgtgcacgttcatattctgccgcgtaaagcgggcgacttccatcgtaatgattctatctatgatgcattggaaaagcacgatcgtgaagacaaagactccccggcactgtggcgtagcgaagaggagatggctgctgaagcggcggcgttacgtgtgtacttccaa

SEQ ID NO: 5 - DNA, 639 bp, synthetic construct
  source 1..639: mol_type = other DNA
atgtccactaatatcaccttccatgcctctgcactgactcgttcggaacgtacagaattacgcaatcagcgcggccttacaatctggctgactgggctgtcggcatcgggtaagtcaactttggccgtagaattagagcaccaacttgttcgtgaccgcggcgtacacgcctatcgtcttgatggagataacattcgtttcggacttaacaaggacttaggtttcagcgaggcggaccgcaatgaaaacattcgccgtatcgcggaagtggcaaaacttttcgccgactccaatagcatcgcaattacttcttttattagtccatatcgtaaggatcgcgatacagcgcgtcagcttcacgaggtagcgacacctggggaggagaccgggctgccgtttgttgaggtttacgttgacgtgcccgtggaggtagcagaacaacgtgacccgaaaggactttataagaaggcccgtgaaggagtgatcaaggaatttacgggcatttccgcaccttatgaagctccagctaaccctgaagtacacgttaaaaattacgaattacccgtccaagatgctgtgaaacaaattattgactatctggattctaagggctacctgcctgcgaagaaggagctcgag

SEQ ID NO: 6 - DNA, 1008 bp, synthetic construct
  source 1..1008: mol_type = other DNA
atggcggaagatcgtgccatgcctgttatgcctccggcagcggatgcggcagaggctgtcccagccgccccgacagccttgcctgaggaaggacctgcaggaccggaagcaccattgcaaacactgcacgggccacgccactttccggcggttgatgccaatgccatccgtcaggcctttgaggggggtcattatccctacccgcgtcgcttaggccgcgtggtctatgaggcagaaaaggcacgtttacaagctgaactgttgaaagttcagatttgggcccaggaaacaggtcaaaagttcgtcattcttatggaaggtcgcgatgctgcgggcaagggaggaaccatcaaacgctttatggagcatttgaatccccgttacgcgcgcgtcgttgcgttgaccaagcctggtgaacgcgagcgtggtcagtggtttttccaacgttacatcgaacatttacccactgcaggagaaatcgtcttctttgaccgctcctggtataatcgtgctggcgtggagcgcgtaatggggttttgtaccccgtccgaatacctggaattcatgcgtcaggcaccggaacttgaacgtatgttggttcgctctggcattcgcttatataaatattggttcagtgtgacacgtgatgagcaacgtgcgcgttttttagcgcgcgaaaccgacccgctgaagcgctggaagttatcaccgatcgtaaaagctagcttagataagtgggatgattacactgaggctaaggaggctatgtttttttacaccgatacggcggatgccccctggaccattgtgaaatctaacgacaaaaagcgtgctcgcttgaattgtatgcgccacttcttatcttcgttggattatccgggaaaggaccccgaagtggtcggtgttcccgaccctttgatcgtgggtcgcgctgcccaagttatcggaactgcagcagatatcttggattctgctaccccccctgcattgcgtaagccacgtcagggt

SEQ ID NO: 7 - DNA, 447 bp, synthetic construct
  source 1..447: mol_type = other DNA
atgtcatttaggttcggacaaatgctaataaagccgagcgttgtctttctgaaaaccgaactgagcttcgccttggttaaccgcaagccggttgttccgggtcatgtgctggtgtgcccgctgagacctgttgaacgttttcgcgacatgagcccggaggaggttgcagacttgtttcaagcggcgcagcgcgtgggcacggtggtcgagaagcacttccaaggtacttcgctgaccttttccatgcaggatggtccagaggccggccagaccgtaaaacacgtgcacgttcatattctgccgcgtaaagcgggcgacttccatcgtaatgattctatctatgatgcagatgaaaagcacgatcgtgaagacaaagactccccggcactgtggcgtagcgaagaggagatggctgctgaagcggcggcgttacgtgtgtacttccaa

SEQ ID NO: 8 - DNA, 735 bp, synthetic construct
  source 1..735: mol_type = other DNA
atgtccaaactcgatctaaacgccctgaacgaactgccgaaggtagatcgcattctggcgctggcggaaactaacgcccaactggaaaaactggacgctgaaggccgcgtagcctgggcgctggataatctgcccggtgaatatgtgctttcttccagcttcggcattcaggcggcggtgagcctgcatctggtgaatcagattcggccggatattccagtgatcctcaccgataccggttatctgtttccggaaacctaccgctttattgacgagttaacggacaaactcaagctcaacctgaaagtgtaccgtgctaccgaaagcgccgcctggcaggaagcacgctacggcaaactgtgggaacagggcgttgaaggcattgaaaagtacaatgacatcaacaaagtcgaaccgatgaaccgggcgctgaaagaacttaacgcgcaaacctggtttgctggcctgcgccgcgaacaatccggcagccgcgccaatttaccggtgctggcaattcagcgtggcgtatttaaagtgctgccgattatcgactgggataaccgaactatttatcagtacctgcaaaaacatggcctgaaatatcacccattatgggatgaaggatatttatcggtcggtgatacccatacaacccgtaaatgggaacctggcatgtcggaagaagaaacacgtttctttggcttaaaaagggaatgtggattgcacgaagggtaa

SEQ ID NO: 9 - protein, 322 aa, synthetic construct [sequence omitted]

SEQ ID NO: 10 - protein, 317 aa, synthetic construct
MSNEYGDKNLKIFSLNSNPELAKEIADNVGVQLGKCSVTRFSDGEVQINIEESIRGCDCYIIQSTSAPVNEHIMELLIMVDALKRASAKTINIVIPYYGYARQDRKARSREPITAKLFANLLETAGATRVIALDIHAPQIQGFFDIPIDHLMGVPILGHYFEGKDLKDIVIVSPDHGGVTRARKLADRLKAPIAIIDKRRPRPNEVEVMNIVGNVEGKTAILIDDIIDTAGTITLAANALVENGAAEVYACCTHPVLSGPAVERINNSKIKELVVTNSIKLPEEKKIERFKQLSVGPLLAEAIIRVHEKQSVSYLFS

SEQ ID NO: 11 - protein, 188 aa, synthetic construct
MSDDRINYLKNKLVQYPDFPKKGILFEDIMPIFQDPRAFGILIDLLLEAVETEFNNIDVIVGLEARGFLFGPTLALRANCAFVPVRKPNKLPGDLVVVSYNKEYSTDSFAIQKGTIKPGQRVLIVDDILATGGTALAADELVTRLGGELVGHLFLLELTFLQGRKRLMAPTYTLLTGQDEAPDGSEFA

SEQ ID NO: 12 - protein, 149 aa, synthetic construct
MSFRFGQHLIKPSVVFLKTELSFALVNRKPVVPGHVLVCPLRPVERFRDMSPEEVADLFQAAQRVGTVVEKHFQGTSLTFSMQDGPEAGQTVKHVHVHILPRKAGDFHRNDSIYDALEKHDREDKDSPALWRSEEEMAAEAAALRVYFQ

SEQ ID NO: 13 - protein, 213 aa, synthetic construct
MSTNITFHASALTRSERTELRNQRGLTIWLTGLSASGKSTLAVELEHQLVRDRGVHAYRLDGDNIRFGLNKDLGFSEADRNENIRRIAEVAKLFADSNSIAITSFISPYRKDRDTARQLHEVATPGEETGLPFVEVYVDVPVEVAEQRDPKGLYKKAREGVIKEFTGISAPYEAPANPEVHVKNYELPVQDAVKQIIDYLDSKGYLPAKKELE

SEQ ID NO: 14 - protein, 336 aa, synthetic construct
MAEDRAMPVMPPAADAAEAVPAAPTALPEEGPAGPEAPLQTLHGPRHFPAVDANAIRQAFEGGHYPYPRRLGRVVYEAEKARLQAELLKVQIWAQETGQKFVILMEGRDAAGKGGTIKRFMEHLNPRYARVVALTKPGERERGQWFFQRYIEHLPTAGEIVFFDRSWYNRAGVERVMGFCTPSEYLEFMRQAPELERMLVRSGIRLYKYWFSVTRDEQRARFLARETDPLKRWKLSPIVKASLDKWDDYTEAKEAMFFYTDTADAPWTIVKSNDKKRARLNCMRHFLSSLDYPGKDPEVVGVPDPLIVGRAAQVIGTAADILDSATPPALRKPRQG

SEQ ID NO: 15 - protein, 149 aa, synthetic construct
MSFRFGQMLIKPSVVFLKTELSFALVNRKPVVPGHVLVCPLRPVERFRDMSPEEVADLFQAAQRVGTVVEKHFQGTSLTFSMQDGPEAGQTVKHVHVHILPRKAGDFHRNDSIYDADEKHDREDKDSPALWRSEEEMAAEAAALRVYFQ

SEQ ID NO: 16 - DNA, 930 bp, synthetic construct
  source 1..930: mol_type = other DNA
atgcaaaacgcaggcagcctcgttgttcttggcagcattaatgctgaccacattcttaatcttcaatcttttcctactccaggcgaaaccgtaaccggtaaccactatcaggttgcatttggcggcaaaggcgcgaatcaggctgtggctgctgggcgtagcggtgcgaatatcgcgtttattgcctgtacgggtgatgacagcattggtgagagcgttcgccagcagctcgccactgataacattgatattactccggtcagcgtgatcaaaggcgaatcaacaggtgtggcgctgatttttgttaatggcgaaggtgagaatgtcatcggtattcatgccggcgctaatgctgccctttccccggcgctggtggaagcgcaacgtgagcgtattgccaacgcgtcagcattattaatgcagctggaatcaccactcgaaagtgtgatggcagcggcgaaaatcgcccatcaaaataagactatcgttgcgcttaacccggctccggctcgcgaacttcctgacgaactgctggcgctggtggacattattacgccaaacgaaacggaagcagaaaagctcaccggtattcgtgttgaaaatgatgaagatgcagcgaaggcggcgcaggtactgcatgaaaaaggtatccgtactgtactgattactttaggaagtcgtggtgtatgggctagcgtgaatggtgaaggtcagcgcgttcctggattccgggtgcaggctgtcgataccattgctgccggagatacctttaacggtgcgttaatcacggcattgctggaagaaaaaccattgccagaggcgattcgttttgcccatgctgccgctgcgattgccgtaacacgtaaaggcgcacaaccttccgtaccgtggcgtgaagagatcgacgcatttttagacaggcagaggtga

SEQ ID NO: 17 - DNA, 1002 bp, synthetic construct
  source 1..1002: mol_type = other DNA
atgggtattacagtaataggttctctaaactatgatttggacacatttacggatagattacctaacgctggagaaactttcagggctaaccacttcgaaacacatgctggtggtaagggattgaaccaagctgcggccattggtaaattaaaaaaccccagcagcagatatagtgttcgaatgattggtaatgttggaaatgatacatttggtaaacaattgaaggacactttatccgattgcggagtcgatatcactcacgtcggtacttacgaaggcattaatacgggtaccgctaccatactaattgaagagaaagctggtggccaaaataggatattgattgtagaaggtgctaacagcaagactatttatgacccgaaacagttgtgtgaaatttttccagagggcaaggaggaagaagagtatgttgtttttcaacacgaaattcctgatcctctttccattattaaatggatacatgcgaacaggccgaattttcagatcgtatataacccctcacctttcaaggccatgcctaagaaagattgggagttggtagaccttttggtcgttaatgaaattgagggtcttcaaatcgtggaaagtgtatttgataatgaacttgttgaagaaataagggagaagataaaggacgactttttaggagaatatcgtaaaatttgtgagcttttgtatgaaaaactcatgaatcgaaagaaaagaggaattgtggttatgactttgggttcgagaggggtgcttttctgttcgcacgaaagccctgaagtacaattccttccggctattcaaaatgtttcggttgttgatactacaggagctggagatactttcctgggcggtttggttactcaattgtatcaaggagagaccttgtctacggctataaagttctctacattagctagttcattgaccattcaaagaaaaggtgctgctgaaagcatgccactgtataaagatgttcagaaagatgcataa

SEQ ID NO: 18 - DNA, 948 bp, synthetic construct
  source 1..948: mol_type = other DNA
gtgcctgatatgaagctttttgctggtaacgccaccccggaactagcacaacgtattgccaaccgcctgtacacttcactcggcgacgccgctgtaggtcgctttagcgatggcgaagtcagcgtacaaattaatgaaaatgtacgcggtggtgatattttcatcatccagtccacttgtgcccctactaacgacaacctgatggaattagtcgttatggttgatgccctgcgtcgtgcttccgcaggtcgtatcaccgctgttatcccctactttggctatgcgcgccaggaccgtcgcgtccgttccgctcgtgtaccaatcactgcgaaagtggttgcagacttcctctccagcgtcggtgttgaccgtgtgctgacagtggatctgcacgctgaacagattcagggtttcttcgacgttccggttgataacgtatttggtagcccgatcctgctggaagacatgctgcagctgaatctggataacccaattgtggtttctccggacatcggcggcgttgtgcgtgcccgcgctatcgctaagctgctgaacgataccgatatggcaatcatcgacaaacgtcgtccgcgtgcgaacgtttcacaggtgatgcatatcatcggtgacgttgcaggtcgtgactgcgtactggtcgatgatatgatcgacactggcggtacgctgtgtaaagctgctgaagctctgaaagaacgtggtgctaaacgtgtatttgcgtacgcgactcacccgatcttctctggcaacgcggcgaacaacctgcgtaactctgtaattgatgaagtcgttgtctgcgataccattccgctgagcgatgaaatcaaatcactgccgaacgtgcgtactctgaccctgtcaggtatgctggccgaagcgattcgtcgtatcagcaacgaagaatcgatctctgccatgttcgaacactaa

SEQ ID NO: 19 - DNA, 981 bp, synthetic construct
  source 1..981: mol_type = other DNA
ttgagccacgactggaccgataatcgcaaaaacctgatgctctttgccggccgcgcccatccggagctggccgagcaggtagccaaagagctcgacgtccacgtcacctctcaggacgcgcgggagttcgccaacggcgagatcttcgtgcgcttccacgaatcggtacgcggttgcgacgccttcgtcctgcaatcctgcccggcaccggtgaacaggtggctgatggaacagctgatcatgatcgacgcgctcaaacggggcagcgccaaacggatcaccgccgtcatgccgttctatccgtatgcccggcaagacaagaaacaccgtggccgcgaaccgatctccgcgcgactgatcgccgacctgctcaagaccgcgggcgccgaccggatcgtgacggtcgacctgcacaccgaccagatccagggtttcttcgacgggccggtcgatcatatgcgcggtcagaacctgctgaccggttacatcagagacaactacccggacggcaacatggtggtcgtctcccctgactccggccgggtacgcatcgccgagaagtgggccgacgcattaggtggtgttcccctcgccttcatccacaagacccgtgatccgcgggtacccaaccaagtggtgtccaaccgcgtcgtcggcgacgtggccgggcgcacctgtgtcctgattgacgacatgatcgacaccggcggcaccatcgccggcgcggtggcattgctgcacaacgacggcgccggtgacgtgatcatcgcggcaacccacggcgtgctctccgaccccgctgcgcagcggctggcctcctgcggcgcccgcgaagtgatcgtcacgaacacgctcccgatcggcgaagacaagcgcttcccccagctcaccgttttgtccatcgcgccgctgctggccagcacaatccgggcggtcttcgaaaacggctcagtaacaggacttttcgacggggacgcatag

SEQ ID NO: 20 - DNA, 2333 bp, synthetic construct
  source 1..2333: mol_type = other DNA
tatatatctctaaatgtaaccataacattgtggataataatctgttggagtgtgaacactctgaaagattctaaatcatccaattttatgtgaagaaatatgtagaaaaaacaaacaatggatgagttataggtttggttaagacttgaaaagtcattgatcacggcttttgtccattattccgtgtttaaagtaatttacattctcttttaagatctctcagtccaaagacaatgcaaacaataataatttctccacttgtttctcatcgtctctgtcttgctcgtgctgttccttgcaaccgtcttctcaacaaccaccaccgtgctcctccttcgatccgcctctcaaaccaccgttcaaccacctcactccgcctcttctcctccgccggtagaccccaccaccttctatttcccttttaaactttttgtttccttttgaaaaagatattttgcttgcgtggggcaattccgtcaaagcgttaccattcgggataaattggtcattttttcatcactgtgtctctctattgagtctttagggtctctctgtgtttgcaaatctgacgccgagtattttgctaaagcagcagccagtcgggacagtgaaatggcgactgaagatgtgcaagatcccagaatcgctaagattgcctcttccattagagtcatccccgacttccctaaaccaggtctccattttttccatttataccgattaatatcgtctctagtcttgatctgcgttgggatcttaaggttttaatttggatacttggcaggaatcatgtttcaggacataacgacgcttcttctcgacactgaggcctttaaggatactattgctttgtttgttgatagatacaaagataaaggcatatctgttgttgcaggtaacaactcgtatggttctgtttggtactccaatcgattaacagcttgtggatttagtttcttttggcctttttagatactgagtgctttagcttaaactgaaaagttgaaatgaattggtggatgatttttggttctgcctaaattgatgttgtggtaaatgcaggtgttgaagctagaggtttcatttttggccctcctattgcgttggctattggtgccaaatttgttcccatgaggaagcccaagaagctacctggtacatttttatttcttcctctagttgcttgtgattttgacattaagttacatgaaaggtctcaattaggaagttgttagtttaaatggcttcttgctccaagtctacactttatcattatctcgaacattagcaaggtgacttttatcttgcagtgtctttgatcaatatgatgctactcatttgctagatcatttttctttgtggtaacttgcagtaacttaagtggttgttattttttctgttacatatcaatggacttagttgtgtatcttgaccagttacaagaaatgtattaagccatttggaaacttatgagcagtgtcaaataattgtcgcattccttgtttatccatggacgttaatttctaaactgcctctgcatttggttcctttaacagggaaggttatttcggaggagtattcgttggagtatggaacagatacgattgagatgcacgtaggtgcagtagagcctggtgagcgtgctattattattgatgacctcattgccacgggtgggactctcgctgctgcaatccgactacttggtaagtccaagatttgataatgcagcatatgcagttcagttaaacttgactatttctcactcccacaattggttggttttgcagaacgagtaggagtgaagattgttgaatgtgcttgcgtaattgagttaccagagcttaaggtttagcttatttatctctgttttactatctgcttccaatctcttacactgatttgcttataaagagatatgtgggctgcaatggttttgtagggaaaggagaaactaggagagacgtcgctatttgttcttgtaaagtcggctgcttaacaagaaactggaagagaaggttattggatcgagtgttgatgctatttttcatgtatggtgagacattttgcgtgggatttgatccttgttgtttcaacttatcataattggttcagactagaaaatggcatttgaatgtcaggattcgattgcagtttcctattgttccacttaaaatccccgattaataaggtgttatgggttttatttatagaatcaagttgtaatagtagactccaacaggggctaaagtcttggtaacgcaattcttcttttaagtttcgcagcaccttgctcgttacttctcttctcaactcgtaaggacacaaatcac

SEQ ID NO: 21 - DNA, 552 bp, synthetic construct
  source 1..552: mol_type = other DNA
atgaccgcgactgcacagcagcttgagtatctcaaaaatagcatcaaaagcattcaggactacccaaaacccggcattcttttccgcgatgtcaccagcttactggaagacccgaaagcttacgctctcagcatcgacttgctggttgagcgttacaaaaatgcgggcattaccaaagttgtcggcaccgaagcgcgtggcttcttgtttggcgctccggtagctctgggtctgggcgttggctttgtaccggtccgtaaaccgggcaaactgccgcgtgaaaccatcagtgaaacttacgacctggaatacggcaccgatcagctggagatccacgttgatgccatcaaaccgggcgacaaagttctggtggtggacgacctgctggcaaccggcggcactatcgaagcgaccgttaaactgatccgtcgtctgggtggtgaagtggctgacgctgcgttcattatcaacctgttcgatctcggcggcgaacagcgtctcgaaaaacagggcattaccagctacagccttgtcccgttcccgggccattaa

SEQ ID NO: 22 - DNA, 606 bp, synthetic construct
  source 1..606: mol_type = other DNA
atggcgctgcatgacgaaaacgtcgtctggcatagccatccggtcactgtgcaacaacgcgagctacaccacggtcatcgtggtgtagtgctgtggtttaccggcctctccgggtccggtaaatcaacggtcgccggggcgctggaggaggcgttacataaactcggcgtcagtacgtatctgctggatggcgacaatgttcgccacggattatgcagcgatctcggttttagcgatgccgatcgtaaagagaatatccgtcgcgtcggtgaagtggcgaatttgatggttgaagccggactggtggtgctgaccgcatttatctcgccacaccgcgccgaacgccagatggttcgcgaacgcgtaggagaagggcgctttatcgaagtgtttgtcgatacgccgctggcgatttgcgaagcccgcgatcccaaaggcttatataagaaagcgcgtgccggtgaactgcgcaactttacgggaatagattccgtttacgaagcgcctgaatcggcagaaattcatctcaatggtgaacaattagtaacaaatttggtacagcaattattagatctgttgagacagaacgatattatcagatcctga

SEQ ID NO: 23 - DNA, 609 bp, synthetic construct
  source 1..609: mol_type = other DNA
atggctactaatattacttggcatccaaatcttacttacgacgaacgcaaggcattgagaaaacaggacggttgtactatttggttaacaggtctaagtgcgtcaggtaaaagtacaatcgcctgtgcgctagaacagttactgctccaaaaaaacttgtctgcatatagattggatggtgacaacattcgttttggattgaacaaggatttgggtttctcagaaaaggacagaaatgaaaacattcgtagaattagcgaagtttctaagctatttgctgattcatgtgctatttcaatcacctcatttatctctccatacagagttgacagagatagagctcgtgaactacataaggaggctggtttgaagttcattgaaatatttgttgatgttccattagaagtcgctgagcaaagggaccctaagggtttatacaagaaagctagggagggtgtaatcaaggagtttacaggtatttctgccccatatgaagcgccaaaagctccagagctacatttgagaaccgaccagaagacggttgaagaatgtgctaccattatttatgagtacttaatcagtgaaaaaatcatccgtaagcatttgtaa

SEQ ID NO: 24 - DNA, 2067 bp, synthetic construct
  source 1..2067: mol_type = other DNA
atgggtcaggaaaagctatacatcgaaaaagagctcagttggttatcgttcaatgaacgcgtgcttcaggaagcggcggacaaatctaacccgctgattgaaaggatgcgtttcctggggatctattccaataaccttgatgagttctataaagtccgcttcgctgaactgaagcgacgcatcattattagcgaagaacaaggctccaactctcattcccgccatttactgggcaaaattcagtcccgggtgctgaaagccgatcaggaattcgacggcctctacaacgagctattgctggagatggcgcgcaaccagatcttcctgattaatgaacgccagctctccgtcaatcaacaaaactggctgcgtcattattttaagcagtatctgcgtcagcacattacgccgattttaatcaatcctgacactgacttagtgcagttcctgaaagatgattacacctatctggcggtggaaattatccgtggcgataccatccgttacgcgctgctggagatcccatcagataaagtgccgcgctttgtgaatttaccgccagaagcgccgcgtcgacgcaagccgatgattcttctggataacattctgcgttactgccttgatgatattttcaaaggcttctttgattatgacgcgctgaatgcctattcaatgaagatgacccgcgatgccgaatacgatttagtgcatgagatggaagccagcctgatggagttgatgtcttccagtctcaagcagcgtttaactgctgagccggtgcgttttgtttatcagcgcgatatgcccaatgcgctggttgaagtgttacgcgaaaaactgactatttcccgctacgactccatcgtccccggcggtcgttatcataattttaaagactttattaatttccccaatgtcggcaaagccaatctggtgaacaaaccactgccgcgtttacgccatatttggtttgataaagcccagttccgcaatggttttgatgccattcgcgaacgcgatgtgttgctctattatccttatcacacctttgagcatgtgctggaactgctgcgtcaggcttcgttcgacccgagcgtactggcgattaaaattaacatttaccgcgtggcgaaagattcacgcatcatcgactcgatgatccacgccgcacataacggtaagaaagtcaccgtggtggttgagttacaggcgcgtttcgacgaagaagccaacattcactgggcgaagcgcctgaccgaagcaggcgtgcacgttatcttctctgcgccggggctgaaaattcacgccaaactgttcctgatttcacgtaaagaaaacggtgaagtggtgcgttatgcacacatcgggaccgggaactttaacgaaaaaaccgcgcgtctttatactgactattcgttgctgaccgccgatgcgcgcatcaccaacgaagtacggcgggtatttaactttattgaaaacccataccgtccggtgacatttgattatttaatggtatcgccgcaaaactcccgccgcctattgtatgaaatggtggaccgcgagatcgccaacgcgcagcaagggctgcccagtggtatcaccctgaagctaaataaccttgtcgataaaggcctggttgatcgtctgtatgcggcctccagctccggcgtaccggttaatctgctggttcgcggaatgtgttcgctgatccccaatctggaaggcattagcgacaacattcgtgccatcagtattgttgaccgttaccttgaacatgaccgggtttatatttttgaaaatggcggcgataaaaaggtctacctttcttccgccgactggatgacgcgcaatattgattatcgtattgaagtggcgacgccgctgctcgatccgcgcctgaagcagcgggtactggacatcatcgacatattgttcagcgatacggtcaaagcacgttatatcgataaagaactcagtaatcgctacgttccccgcggcaatcgccgcaaagtacgggcgcagttggcgatttatgactacatcaaatcactcgaacaacctgaataa

SEQ ID NO: 25 - DNA, 56 bp, synthetic construct
  source 1..56: mol_type = other DNA
aggcaaggcaaacagtgaggaatcttaatacgactcactataggggaattgtgagc

SEQ ID NO: 26 - DNA, 47 bp, synthetic construct
  source 1..47: mol_type = other DNA
aggcgtttaatttatccggcaatatcaaaaaacccctcaagacccgt

SEQ ID NO: 27 - DNA, 23 bp, synthetic construct
  source 1..23: mol_type = other DNA
tgtcagcgatgagcatatcgtga

SEQ ID NO: 28 - DNA, 48 bp, synthetic construct
  source 1..48: mol_type = other DNA
aattcccctatagtgagtcgtattaagattcctcactgtttgccttgc

SEQ ID NO: 29 - DNA, 19 bp, synthetic construct
  source 1..19: mol_type = other DNA
gcgtcgagatcctgctcct

SEQ ID NO: 30 - DNA, 38 bp, synthetic construct
  source 1..38: mol_type = other DNA
agtcgtattaattgaaatctccggcttgaaacaaatgt

SEQ ID NO: 31 - DNA, 26 bp, synthetic construct
  source 1..26: mol_type = other DNA
gggttttttgcgacgcagaaggcggc

SEQ ID NO: 32 - DNA, 19 bp, synthetic construct
  source 1..19: mol_type = other DNA
attcgcgaatcgccgttcc

SEQ ID NO: 33 - DNA, 31 bp, synthetic construct
  source 1..31: mol_type = other DNA
taatacgactcactataggggaattgtgagc

SEQ ID NO: 34 - DNA, 20 bp, synthetic construct
  source 1..20: mol_type = other DNA
ttattgctcagcggtggcag

SEQ ID NO: 35 - DNA, 51 bp, synthetic construct
  source 1..51: mol_type = other DNA
ctgccaccgctgagcaataataatacgactcactataggggaattgtgagc

SEQ ID NO: 36 - DNA, 20 bp, synthetic construct
  source 1..20: mol_type = other DNA
atctcagtggtggtggtggt

SEQ ID NO: 37 - DNA, 18 bp, synthetic construct
  source 1..18: mol_type = other DNA
taatacgactcactatag

SEQ ID NO: 38 - DNA, 687 bp, synthetic construct
  source 1..687: mol_type = other DNA
atgcgtccagcaggccgtagcaataatcaggtgcgtcccgttaccctgactcgtaactatacaaaacatgcagaaggctcggtgctggtcgaatttggcgataccaaagtgttgtgtaccgcctctattgaagaaggcgtgccgcgcttcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgaggggaatcgaatccattgtag

(19) United States
(12) Patent Application Publication
(10) Pub. No.: US 2026/0308651 A1
(43) Pub. Date: Oct. 8, 2026

(54) NOVEL BIOSYNTHESIS SYSTEM FOR PRODUCING 3'-PHOSPHOADENOSINE-5'-PHOSPHOSULFATE AND APPLICATION THEREOF

(71) Applicant: Jiangnan University, Wuxi (CN)

(72) Inventors: Jing Wu, Wuxi (CN); Simin Gu, Wuxi (CN); Xiaomin Li, Wuxi (CN); Liming Liu, Wuxi (CN); Hejia Qi, Wuxi (CN); Lingxin Huang, Wuxi (CN); Xiangting Song, Wuxi (CN); Wei Song, Wuxi (CN); Wanqing Wei, Wuxi (CN); Guipeng Hu, Wuxi (CN); Jia Liu, Wuxi (CN)

(21) Appl. No.: 19/681,666

(22) Filed: May 19, 2026

(30) Foreign Application Priority Data
Jun. 27, 2025 (CN) 2025108766549

(51) Int. Cl. C12P 19/32; C12N 1/20; C12N 9/10; C12N 9/12; C12N 15/70; C12N 15/75; C12R 1/125; C12R 1/19

(52) U.S. Cl. CPC C12P 19/32; C12N 1/20; C12N 9/1077; C12N 9/1205; C12N 9/1229; C12N 9/1235; C12N 9/13; C12N 15/70; C12N 15/75; C12R 2001/125; C12R 2001/19; C12Y 204/02007; C12Y 207/01015; C12Y 207/01025; C12Y 207/04; C12Y 207/06001; C12Y 208/02

(57) ABSTRACT

The present disclosure discloses a novel biosynthesis system for producing 3′-phosphoadenosine-5′-phosphosulfate and an application thereof, and belongs to the technical field of bioengineering. The present disclosure constructs an in vitro six-enzyme cascade reaction using ribose and adenosine as substrates by recombinantly expressing and purifying RBKS, PRS, APRT, APSST, APSK, and PPK2, achieves a molar conversion rate of 22.85%, and constructs PAPS-producing cell factories of Escherichia coli and Bacillus subtilis, with PAPS yields reaching 7.60 g/L and 5.03 g/L respectively through 5-L fermenter fermentation. The PAPS synthesis system provided by the present disclosure offers a scalable and cost-effective solution for production of sulfated compounds.

### REFERENCE TO SEQUENCE LISTING

The instant application contains a Sequence Listing in XML format as a file named “PC260003A-SEQ.xml”, created on May 11, 2026, of 52,304 bytes in size, and which is hereby incorporated by reference in its entirety.

### TECHNICAL FIELD

The present disclosure relates to a novel biosynthesis system for producing 3′-phosphoadenosine-5′-phosphosulfate and an application thereof, and belongs to the technical field of bioengineering.

### BACKGROUND

3′-phosphoadenosine-5′-phosphosulfate (PAPS) is a high-energy compound with a molecular formula of C₁₀H₁₄NO₁₃P₂S and a molecular weight of 507.27 g·mol⁻¹, and has two phosphate groups and one sulfate group attached to 3′ and 5′ positions of adenosine, respectively. In a reductase-catalyzed reaction, a high-energy sulfate bond is readily cleaved to release a sulfate group, such that a sulfite compound or a sulfur-containing compound is formed, and synthesis and sulfation modification of amino acids, proteins, hormones, flavonoids, and other compounds are completed in vivo. PAPS exhibits good solubility in water and relative stability under physiological conditions (37° C., pH 7.4), with a half-life of approximately 24 hours. PAPS can be stored for a long time at low temperatures (below 0° C.) without significant degradation, with the half-life of only a few minutes at 60° C., decomposes almost instantly at 80° C., and readily hydrolyzes under acidic or alkaline conditions to form adenosine monophosphate and inorganic sulfate, thereby resulting in loss of biological activity.

Currently, a double-enzyme method achieves a high conversion rate by using adenosine triphosphate (ATP) as a substrate to synthesize PAPS, but due to high cost and easy degradation of ATP, the double-enzyme method is relatively cumbersome in implementation and is unsuitable for large-scale production; biosynthesis based on a native PAPS synthesis system is limited by ATP supply and has a low yield; and no studies on PAPS-producing cell factories have been reported domestically or internationally. Both de novo synthesis cell factories and whole-cell catalytic systems for functional glycosaminoglycan derivatives such as heparan sulfate and chondroitin sulfate suffer from insufficient production performance, low synthesis efficiency, and high production costs due to limitation in PAPS supply. Therefore, it is necessary to explore a novel, high-efficiency, low-cost PAPS biosynthesis strategy for efficient PAPS production and supply, so as to provide a new idea for constructing cell factories for sulfated compounds, which has important scientific significance and application value in expanding a sulfotransferase resource library, and promoting the development of enzyme engineering and metabolic engineering.

### SUMMARY

The present disclosure provides a novel PAPS biosynthesis system, and the system with ribose and adenosine as substrates is used to synthesize PAPS via a six-enzyme cascade reaction catalyzed by ribokinase (RBKS), phosphoribosyl pyrophosphate synthetase (PRS), adenine phosphoribosyltransferase (APRT), adenine sulfotransferase (APSST), adenosine 5′-phosphosulfate kinase (APSK), and polyphosphate kinase 2 (PPK2).

A first object of the present disclosure is to provide a novel biosynthesis system for producing 3′-phosphoadenosine-5′-phosphosulfate, and the system includes the following enzymes: ribokinase (RBKS), phosphoribosyl pyrophosphate synthetase (PRS), adenine phosphoribosyltransferase (APRT), adenine sulfotransferase (APSST), adenosine 5′-phosphosulfate kinase (APSK), and polyphosphate kinase 2 (PPK2).

In an embodiment of the present disclosure, RBKS is derived from Homo sapiens (human); PRS is derived from Bacillus amyloliquefaciens; APRT is derived from Schizosaccharomyces pombe; APSST is derived from Bos Taurus (bovine); APSK is derived from Penicillium chrysogenum; and PPK2 is derived from Rhodobacter sphaeroides.

In an embodiment of the present disclosure, a nucleotide sequence of a gene encoding RBKS is as shown in SEQ ID NO: 1; a nucleotide sequence of a gene encoding PRS is as shown in SEQ ID NO: 2; a nucleotide sequence of a gene encoding APRT is as shown in SEQ ID NO: 3; a nucleotide sequence of a gene encoding APSST is as shown in SEQ ID NO: 4 or SEQ ID NO: 7; a nucleotide sequence of a gene encoding APSK is as shown in SEQ ID NO: 5; and a nucleotide sequence of a gene encoding PPK2 is as shown in SEQ ID NO: 6.

In the present disclosure, APSST may preferably be a variant APSST* in which histidine at position 8 is mutated to methionine and leucine at position 117 is mutated to aspartic acid, and a nucleotide sequence of the mutant is as shown in SEQ ID NO: 7.

A second object of the present disclosure is to provide an application of the biosynthesis system in producing 3′-phosphoadenosine-5′-phosphosulfate, and specifically 3′-phosphoadenosine-5′-phosphosulfate is obtained by catalyzing a reaction where enzymes in the biosynthesis system are used as catalysts, and ribose and adenosine serve as substrates.

In an embodiment of the present disclosure, amounts of the enzymes added in the biosynthesis system are as follows: an amount of RBKS added is 0.005-0.015 mM, an amount of PRS added is 0.01-0.03 mM, an amount of APRT added is 0.05-0.07 mM, an amount of APSST added is 0.05-0.15 mM, an amount of APSK added is 0.05-0.15 mM, and an amount of PPK2 added is 0.05-0.15 mM.

In an embodiment of the present disclosure, an amount of ribose added is 1-10 mM, and an amount of adenosine added is 1-10 mM.

In an embodiment of the present disclosure, the catalytic reaction is performed at 25-35° C. and 200-300 rpm for 10-60 minutes.

A third object of the present disclosure is to provide a nucleic acid vector, and the nucleic acid vector includes coding sequences encoding the following enzymes: RBKS, PRS, APRT, APSST, APSK, and PPK2, where the coding sequences of the enzymes are located in one, two, or more expression cassettes.

A fourth object of the present disclosure is to provide an engineered strain expressing the biosynthesis system, and the engineered strain expresses the following enzymes: RBKS, PRS, APRT, APSST, APSK, and PPK2.

In an embodiment of the present disclosure, engineered strain has a cysH gene knocked out.

In an embodiment of the present disclosure, a host cell of engineered strain is Escherichia coli or Bacillus subtilis.

In an embodiment of the present disclosure, the Escherichia coli is Escherichia coli BL21(DE3), and the Bacillus subtilis is Bacillus subtilis 168.

In an embodiment of the present disclosure, when a host of the engineered strain is Escherichia coli, the engineered strain further includes three copies of a gene encoding APSST as shown in SEQ ID NO: 7 inserted at a cysH gene locus of a genome, and three copies of the gene encoding APSST as shown in SEQ ID NO: 7 inserted at a rph pseudogene locus.

In an embodiment of the present disclosure, when the host of the engineered strain is Bacillus subtilis, the engineered strain further includes six copies of the gene encoding APSST as shown in SEQ ID NO: 7 inserted at the cysH gene locus of the genome.

A fifth object of the present disclosure is to provide an application of the engineered strain in fermentative production of 3′-phosphoadenosine-5′-phosphosulfate, and the application includes de novo synthesis of 3′-phosphoadenosine-5′-phosphosulfate using ribose and adenosine as substrates with the engineered strain.

In an embodiment of the present disclosure, the application specifically includes culturing the engineered strain by fermentation in a medium supplemented with ribose and adenosine.

In an embodiment of the present disclosure, the medium is a Luria-Bertani (LB) medium or a Terrific Broth (TB) medium.

In an embodiment of the present disclosure, the application further includes supplementing ribose and adenosine during fermentation.

In an embodiment of the present disclosure, the fermentation is shake flask fermentation, and fermentation conditions include an inoculum size of 2-5%, an initial OD₆₀₀ of 0.8-1.0, a temperature of 30-38° C., a rotation speed of 220 rpm, and a fermentation period 30-36 hours.

In an embodiment of the present disclosure, the fermentation is fermenter fermentation, and fermentation conditions include an inoculum size of 5-15%, a working volume of 20-60%, an initial OD₆₀₀ of 0.8-1.0, a fermentation temperature of 30-38° C., a rotation speed of 300-700 rpm, a pH controlled at 6.5-7.5, an aeration rate of 1-2 vvm, and a fermentation period of 30-36 hours.

The present disclosure further provides a composition for producing 3′-phosphoadenosine-5′-phosphosulfate, the composition includes enzymes in the above biosynthesis system, and the enzymes include RBKS, PRS, APRT, APSST, APSK, and PPK2; a nucleotide sequence of a gene encoding RBKS is as shown in SEQ ID NO: 1; a nucleotide sequence of a gene encoding PRS is as shown in SEQ ID NO: 2; a nucleotide sequence of a gene encoding APRT is as shown in SEQ ID NO: 3; a nucleotide sequence of a gene encoding APSST is as shown in SEQ ID NO: 4 or SEQ ID NO: 7; a nucleotide sequence of a gene encoding APSK is as shown in SEQ ID NO: 5; and a nucleotide sequence of a gene encoding PPK2 is as shown in SEQ ID NO: 6.

The present disclosure further provides a recombinant host cell containing nucleic acids encoding enzymes in the above biosynthesis system, and the enzymes include RBKS, PRS, APRT, APSST, APSK, and PPK2; a nucleotide sequence of a gene encoding RBKS is as shown in SEQ ID NO: 1; a nucleotide sequence of a gene encoding PRS is as shown in SEQ ID NO: 2; a nucleotide sequence of a gene encoding APRT is as shown in SEQ ID NO: 3; a nucleotide sequence of a gene encoding APSST is as shown in SEQ ID NO: 4 or SEQ ID NO: 7; a nucleotide sequence of a gene encoding APSK is as shown in SEQ ID NO: 5; and a nucleotide sequence of a gene encoding PPK2 is as shown in SEQ ID NO: 6.

The present disclosure further provides a method for fermentatively producing 3′-phosphoadenosine-5′-phosphosulfate using the above engineered strain, and the method includes synthesizing 3′-phosphoadenosine-5′-phosphosulfate using ribose and adenosine as substrates with the engineered strain.

### Beneficial Effects

The present disclosure constructs a novel PAPS biosynthesis system, in vitro multi-enzyme cascade catalysis achieves a molar conversion rate of 22.85%, and the PAPS synthesis system is applied to industrial chassis strains Escherichia coli and Bacillus subtilis to construct PAPS-producing cell factories. Efficient biosynthesis of PAPS in a 5-L fermenter is achieved, PAPS yields reach 7.60 g/L and 5.03 g/L, respectively, and a scalable and cost-effective solution for the production of sulfated compounds is provided for production of sulfated compounds.

### BRIEF DESCRIPTION OF THE DRAWINGS

FIG. 1 is a schematic diagram of a novel PAPS synthesis system.

FIG. 2 is an HPLC-MS chromatogram of PAPS.

FIG. 3 is a schematic diagram of a plasmid pET-Duet-RBKS-PRS-APRT.

FIG. 4 is a schematic diagram of a plasmid pRSF-Duet-APSK*-APSST-PPK2.

FIG. 5 illustrates shake flask fermentation results of recombinant Escherichia coli.

FIG. 6 illustrates fermentation results of recombinant Escherichia coli in a 5-L fermenter.

FIG. 7 is a schematic diagram of a plasmid pTA-RBKS-PRS-APRT.

FIG. 8 is a schematic diagram of a plasmid pHT-APSK*-APSST-PPK2.

FIG. 9 illustrates analysis results of fermentation of recombinant Bacillus subtilis in a 5-L fermenter.

### DETAILED DESCRIPTIONS OF THE EMBODIMENTS

Plasmid construction is performed based on classical molecular biology techniques.

TABLE 1
Vector-related Characteristics

| Plasmid | Description |
|---|---|
| pCas9 | Kan^{R}, repA101 replication origin, araBAD promoter |
| pTargetF | Spe^{R}, sgRNA |
| pET-Duet | Amp^{R}, T7 promoter |
| pRSF-Duet | Kan^{R}, T7 promoter |
| pTA | Chl^{R}, xylA promoter |
| pHT | Kan^{R}, xylA promoter |

Media used in the following examples are as follows:

An LB liquid medium: 10 g/L peptone, 5 g/L yeast extract, and 10 g/L sodium chloride, sterilized at 121° C. for 20 minutes.

An LB solid medium: adding 2% agar to the LB liquid medium.

A TB liquid medium: 2.31 g/L KH₂PO₄, 16.42 g/L K₂HPO₄.3H₂O, 24 g/L yeast extract, 12 g/L peptone, and 4 g/L glycerol.

A detection method used in the following examples is as follows:

Determination of PAPS by high performance liquid chromatography-mass spectrometry (HPLC-MS): An HPLC system equipped with a polyamine column (YMC Pack polyamine II, 250 mm×4.6 mm) is used for analysis and detection. A mobile phase is 0.7 mM KH₂PO₄ filtered and ultrasonically degassed, and a flow rate of the mobile phase is 0.6 mL/minute. Column temperature is maintained at 30° C. throughout the operation, and a detection wavelength is 254 nm.

Sequences involved in the following examples are as follows:

A gene sequence of RBKS

(SEQ ID NO: 1):
atggcagcttcaggagaaccccagaggcaatggcaagaggaagttgctgc
tgtggtggtcgtcggctcctgcatgactgatctcgtgagcttgacctctc
gcctgccaaagacgggtgagacaattcacggccataaattcttcattggt
tttggaggtaagggtgccaaccagtgtgttcaagcggcgcgtctgggtgc
tatgaccagcatggtatgcaaagtgggcaaggacagcttcggcaatgact
acattgaaaacttgaagcaaaacgatatttccaccgagttcacctatcag
accaaagacgccgctactggcaccgcaagcattattgtgaacaacgaagg
tcagaacattatcgtgatcgtggcgggtgcgaatctgctgctcaacaccg
aggacctgcgcgcagcggcgaacgtgatctctcgtgcaaaagtcatggtg
tgccaactggaaatcaccccagcgaccagcctggaagctctcacgatggc
acgccgttcaggtgttaagaccttgtttaacccggctccggccattgcgg
acctggacccgcagttttacaccctgtccgatgttttttgttgtaatgag
agcgaagccgagatcctgaccggcctgacggttggcagtgcagcggacgc
gggcgaagcagccctggttctgctgaaacgtggttgccaggttgttatca
tcacccttggcgcggagggctgcgtggtcctgagccagactgagccggaa
ccgaagcacatcccgaccgagaaagtgaaggcggttgataccaccggtgc
gggtgattcgttcgtgggtgcgctggcgttttacctggcgtattacccga
atttgtctttggaggacatgctgaatcgttccaacttcatcgccgcggtt
agcgttcaagcggcgggtacgcagagcagctatccgtataaaaaagactt
accgctgacgctgttc

An amino acid sequence of RBKS

(SEQ ID NO: 9):
MAASGEPQRQWQEEVAAVVVVGSCMTDLVSLTSRLPKTGETIHGHKFFIG
FGGKGANQCVQAARLGAMTSMVCKVGKDSFGNDYIENLKQNDISTEFTYQ
TKDAATGTASIIVNNEGQNIIVIVAGANLLLNTEDLRAAANVISRAKVMV
CQLEITPATSLEALTMARRSGVKTLFNPAPAIADLDPQFYTLSDVFCCNE
SEAEILTGLTVGSAADAGEAALVLLKRGCQVVIITLGAEGCVVLSQTEPE
PKHIPTEKVKAVDTTGAGDSFVGALAFYLAYYPNLSLEDMLNRSNFIAAV
SVQAAGTQSSYPYKKDLPLTLF

A gene sequence of PRS

(SEQ ID NO: 2):
atgagtaacgaatatggcgataaaaatttgaagattttctctttgaacag
taaccccgaactggcaaaggagattgccgacaacgttggcgtacagcttg
gtaaatgttccgtgacgcgcttcagtgatggtgaggtacaaatcaacatc
gaagaatcaatccgtggatgcgactgttacattattcaatcaacttcagc
acctgtcaatgaacacatcatggagttgttaattatggttgatgcgttga
agcgtgcatctgctaagactatcaacattgttattccatactacggatat
gcccgtcaggatcgcaaggcacgcagccgcgaacccattactgccaagtt
atttgcaaacttgttggaaaccgccggtgccacgcgtgtgatcgctttgg
atattcacgctccgcagattcaggggttcttcgatatcccgatcgaccat
ctgatgggcgtcccaatcctgggtcattattttgaaggcaaggatttaaa
agacatcgtcattgtgtctcctgatcacgggggagtcacgcgcgctcgta
aacttgcagaccgcttgaaagcaccaattgccatcattgacaagcgtcgt
ccccgccccaacgaagtggaggtcatgaatatcgtcggaaatgtagaggg
aaagaccgccatccttattgatgacatcattgatacagcgggcacaatca
cattggctgctaacgcgcttgtggagaatggagcagcggaggtttacgcg
tgctgcactcatccggtactgtccggtccagcggtggagcgcatcaataa
ttctaagatcaaggaattagttgttacaaactcaatcaagttgccagagg
agaagaaaattgaacgtttcaagcaattgagtgtagggcccttactggca
gaagccatcatccgcgtgcacgaaaagcagagtgtctcgtacctgttttc
g

An amino acid sequence of PRS

(SEQ ID NO: 10):
MSNEYGDKNLKIFSLNSNPELAKEIADNVGVQLGKCSVTRFSDGEVQINI
EESIRGCDCYIIQSTSAPVNEHIMELLIMVDALKRASAKTINIVIPYYGY
ARQDRKARSREPITAKLFANLLETAGATRVIALDIHAPQIQGFFDIPIDH
LMGVPILGHYFEGKDLKDIVIVSPDHGGVTRARKLADRLKAPIAIIDKRR
PRPNEVEVMNIVGNVEGKTAILIDDIIDTAGTITLAANALVENGAAEVYA
CCTHPVLSGPAVERINNSKIKELVVTNSIKLPEEKKIERFKQLSVGPLLA
EAIIRVHEKQSVSYLFS

A gene sequence of APRT

(SEQ ID NO: 3):
atgtcagatgacaggataaattatctaaaaaacaagctggtgcagtaccc
ggattttccgaaaaaaggtatcctgttcgaagacatcatgccgatttttc
aggacccgagagcattcggcattctgatcgacctgctgcttgaggccgtt
gaaaccgagttcaacaacatcgatgttattgtgggtctggaggcgcgtgg
ttttctgttcggccctacgttggcgctgcgcgcgaactgcgcgtttgttc
cggttcgtaagccgaataagctgccgggtgatctggtggtggtttcctac
aataaagagtatagcaccgacagcttcgcgattcaaaaaggtacgatcaa
accaggccagcgtgttctgattgtcgatgacatccttgccaccggcggta
cggctctggcggcagatgagctggtcacccgtctgggtggtgaattagtg
ggccacttgttcttgttggagttaacctttctgcaaggtcgtaagcgctt
gatggcaccgacctataccttgctgactggccaagacgaggctccggatg
gctctgagttcgcg

An amino acid sequence of APRT

(SEQ ID NO: 11):
MSDDRINYLKNKLVQYPDFPKKGILFEDIMPIFQDPRAFGILIDLLLEAV
ETEFNNIDVIVGLEARGFLFGPTLALRANCAFVPVRKPNKLPGDLVVVSY
NKEYSTDSFAIQKGTIKPGQRVLIVDDILATGGTALAADELVTRLGGELV
GHLFLLELTFLQGRKRLMAPTYTLLTGQDEAPDGSEFA

A gene sequence of wild-type APSST

(SEQ ID NO: 4):
atgtcatttaggttcggacaacacctaataaagccgagcgttgtctttct
gaaaaccgaactgagcttcgccttggttaaccgcaagccggttgttccgg
gtcatgtgctggtgtgcccgctgagacctgttgaacgttttcgcgacatg
agcccggaggaggttgcagacttgtttcaagcggcgcagcgcgtgggcac
ggtggtcgagaagcacttccaaggtacttcgctgaccttttccatgcagg
atggtccagaggccggccagaccgtaaaacacgtgcacgttcatattctg
ccgcgtaaagcgggcgacttccatcgtaatgattctatctatgatgcatt
ggaaaagcacgatcgtgaagacaaagactccccggcactgtggcgtagcg
aagaggagatggctgctgaagcggcggcgttacgtgtgtacttccaa

An amino acid sequence of wild-type APSST

(SEQ ID NO: 12):
MSFRFGQHLIKPSVVFLKTCLSFALVNRKPVVPGHVLVCPLRPVCRFRDM
SPCCVADLFQAAQRVGTVVCKHFQGTSLTFSMQDGPCAGQTVKHVHVHIL
PRKAGDFHRNDSIYDALCKHDRCDKDSPALWRSCCCMAACAAALRVYFQ

A gene sequence of APSK

(SEQ ID NO: 5):
atgtccactaatatcaccttccatgcctctgcactgactcgttcggaacg
tacagaattacgcaatcagcgcggccttacaatctggctgactgggctgt
cggcatcgggtaagtcaactttggccgtagaattagagcaccaacttgtt
cgtgaccgcggcgtacacgcctatcgtcttgatggagataacattcgttt
cggacttaacaaggacttaggtttcagcgaggggaccgcaatgaaaacat
tcgccgtatcgcggaagtggcaaaacttttcgccgactccaatagcatcg
caattacttcttttattagtccatatcgtaaggatcgcgatacagcgcgt
cagcttcacgaggtagcgacacctggggaggagaccgggctgccgtttgt
tgaggtttacgttgacgtgcccgtggaggtagcagaacaacgtgacccga
aaggactttataagaaggcccgtgaaggagtgatcaaggaatttacgggc
atttccgcaccttatgaagctccagctaaccctgaagtacacgttaaaaa
ttacgaattacccgtccaagatgctgtgaaacaaattattgactatctgg
attctaagggctacctgcctgcgaagaaggagctcgag

An amino acid sequence of APSK

(SEQ ID NO: 13):
MSTNITFHASALTRSCRTCLRNQRGLTIWLTGLSASGKSTLAVCLCHQLV
RDRGVHAYRLDGDNIRFGLNKDLGFSCADRNCNIRRIACVAKLFADSNSI
AITSFISPYRKDRDTARQLHCVATPGCCTGLPFVCVYVDVPVCVACQRDP
KGLYKKARCGVIKCFTGISAPYCAPANPCVHVKNYCLPVQDAVKQIIDYL
DSKGYLPAKKCLE

A gene sequence of PPK2

(SEQ ID NO: 6):
atggcggaagatcgtgccatgcctgttatgcctccggcagcggatgcggc
agaggctgtcccagccgccccgacagccttgcctgaggaaggacctgcag
gaccggaagcaccattgcaaacactgcacgggccacgccactttccggcg
gttgatgccaatgccatccgtcaggcctttgaggggggtcattatcccta
cccgcgtcgcttaggccgcgtggtctatgaggcagaaaaggcacgtttac
aagctgaactgttgaaagttcagatttgggcccaggaaacaggtcaaaag
ttcgtcattcttatggaaggtcgcgatgctgcgggcaagggaggaaccat
caaacgctttatggagcatttgaatccccgttacgcgcgcgtcgttgcgt
tgaccaagcctggtgaacgcgagcgtggtcagtggtttttccaacgttac
atcgaacatttacccactgcaggagaaatcgtcttctttgaccgctcctg
gtataatcgtgctggcgtggagcgcgtaatggggttttgtaccccgtccg
aatacctggaattcatgcgtcaggcaccggaacttgaacgtatgttggtt
cgctctggcattcgcttatataaatattggttcagtgtgacacgtgatga
gcaacgtgcgcgttttttagcgcgcgaaaccgacccgctgaagcgctgga
agttatcaccgatcgtaaaagctagcttagataagtgggatgattacact
gaggctaaggaggctatgtttttttacaccgatacggcggatgccccctg
gaccattgtgaaatctaacgacaaaaagcgtgctcgcttgaattgtatgc
gccacttcttatcttcgttggattatccgggaaaggaccccgaagtggtc
ggtgttcccgaccctttgatcgtgggtcgcgctgcccaagttatcggaac
tgcagcagatatcttggattctgctaccccccctgcattgcgtaagccac
gtcagggt

An amino acid sequence of PPK2

(SEQ ID NO: 14):
MACDRAMPVMPPAADAACAVPAAPTALPCCGPAGPCAPLQTLHGPRHFPA
VDANAIRQAFCGGHYPYPRRLGRVVYCACKARLQACLLKVQIWAQCTGQK
FVILMCGRDAAGKGGTIKRFMCHLNPRYARVVALTKPGCRCRGQWFFQRY
ICHLPTAGCIVFFDRSWYNRAGVCRVMGFCTPSCYLCFMRQAPCLCRMLV
RSGIRLYKYWFSVTRDCQRARFLARCTDPLKRWKLSPIVKASLDKWDDYT
CAKCAMFFYTDTADAPWTIVKSNDKKRARLNCMRHFLSSLDYPGKDPCVV
GVPDPLIVGRAAQVIGTAADILDSATPPALRKPRQ
G

A gene sequence of an APSST mutant (APSST*: H8M/L117D)

(SEQ ID NO: 7):
atgtcatttaggttcggacaaatgctaataaagccgagcgttgtctttct
gaaaaccgaactgagcttcgccttggttaaccgcaagccggttgttccgg
gtcatgtgctggtgtgcccgctgagacctgttgaacgttttcgcgacatg
agcccggaggaggttgcagacttgtttcaagcggcgcagcgcgtgggcac
ggtggtcgagaagcacttccaaggtacttcgctgaccttttccatgcagg
atggtccagaggccggccagaccgtaaaacacgtgcacgttcatattctg
ccgcgtaaagcgggcgacttccatcgtaatgattctatctatgatgcaga
tgaaaagcacgatcgtgaagacaaagactccccggcactgtggcgtagcg
aagaggagatggctgctgaagcggcggcgttacgtgtgtacttccaa

An amino acid sequence of an APSST mutant (APSST*: H8M/L117D)

(SEQ ID NO: 15):
MSFRFGQMLIKPSVVFLKTCLSFALVNRKPVVPGHVLVCPLRPVCRFRDM
SPCCVADLFQAAQRVGTVVCKHFQGTSLTFSMQDGPCAGQTVKHVHVHIL
PRKAGDFHRNDSIYDADCKHDRCDKDSPALWRSCCCMAACAAALRVYFQ

A gene sequence of cysH

(SEQ ID NO: 8):
atgtccaaactcgatctaaacgccctgaacgaactgccgaaggtagatcg
cattctggcgctggcggaaactaacgcccaactggaaaaactggacgctg
aaggccgcgtagcctgggcgctggataatctgcccggtgaatatgtgctt
tcttccagcttcggcattcaggcggcggtgagcctgcatctggtgaatca
gattcggccggatattccagtgatcctcaccgataccggttatctgtttc
cggaaacctaccgctttattgacgagttaacggacaaactcaagctcaac
ctgaaagtgtaccgtgctaccgaaagcgccgcctggcaggaagcacgcta
cggcaaactgtgggaacagggcgttgaaggcattgaaaagtacaatgaca
tcaacaaagtcgaaccgatgaaccgggcgctgaaagaacttaacgcgcaa
acctggtttgctggcctgcgccgcgaacaatccggcagccgcgccaattt
accggtgctggcaattcagcgtggcgtatttaaagtgctgccgattatcg
actgggataaccgaactatttatcagtacctgcaaaaacatggcctgaaa
tatcacccattatgggatgaaggatatttatcggtcggtgatacccatac
aacccgtaaatgggaacctggcatgtcggaagaagaaacacgtttctttg
gcttaaaaagggaatgtggattgcacgaagggtaa

### Example 1: Screening of Enzymes in a Synthesis System

### 1. Screening of RBKS

(1) RBKS derived from Escherichia coli (NCBI No: 948260, SEQ ID NO:16), RBKS derived from Saccharomyces cerevisiae (NCBI No: 850402, SEQ ID NO: 17), and RBKS derived from human as shown in SEQ ID NO: 1 were chemically synthesized;

SEQ ID NO: 16:
atgcaaaacgcaggcagcctcgttgttcttggcagcattaatgctgacca
cattcttaatcttcaatcttttcctactccaggcgaaaccgtaaccggta
accactatcaggttgcatttggcggcaaaggcgcgaatcaggctgtggct
gctgggcgtagcggtgcgaatatcgcgtttattgcctgtacgggtgatga
cagcattggtgagagcgttcgccagcagctcgccactgataacattgata
ttactccggtcagcgtgatcaaaggcgaatcaacaggtgtggcgctgatt
tttgttaatggcgaaggtgagaatgtcatcggtattcatgccggcgctaa
tgctgccctttccccggcgctggtggaagcgcaacgtgagcgtattgcca
acgcgtcagcattattaatgcagctggaatcaccactcgaaagtgtgatg
gcagcggcgaaaatcgcccatcaaaataagactatcgttgcgcttaaccc
ggctccggctcgcgaacttcctgacgaactgctggcgctggtggacatta
ttacgccaaacgaaacggaagcagaaaagctcaccggtattcgtgttgaa
aatgatgaagatgcagcgaaggcggcgcaggtactgcatgaaaaaggtat
ccgtactgtactgattactttaggaagtcgtggtgtatgggctagcgtga
atggtgaaggtcagcgcgttcctggattccgggtgcaggctgtcgatacc
attgctgccggagatacctttaacggtgcgttaatcacggcattgctgga
agaaaaaccattgccagaggcgattcgttttgcccatgctgccgctgcga
ttgccgtaacacgtaaaggcgcacaaccttccgtaccgtggcgtgaagag
atcgacgcatttttagacaggcagaggtga
SEQ ID NO: 17:
atgggtattacagtaataggttctctaaactatgatttggacacatttac
ggatagattacctaacgctggagaaactttcagggctaaccacttcgaaa
cacatgctggtggtaagggattgaaccaagctgcggccattggtaaatta
aaaaaccccagcagcagatatagtgttcgaatgattggtaatgttggaaa
tgatacatttggtaaacaattgaaggacactttatccgattgcggagtcg
atatcactcacgtcggtacttacgaaggcattaatacgggtaccgctacc
atactaattgaagagaaagctggtggccaaaataggatattgattgtaga
aggtgctaacagcaagactatttatgacccgaaacagttgtgtgaaattt
ttccagagggcaaggaggaagaagagtatgttgtttttcaacacgaaatt
cctgatcctctttccattattaaatggatacatgcgaacaggccgaattt
tcagatcgtatataacccctcacctttcaaggccatgcctaagaaagatt
gggagttggtagaccttttggtcgttaatgaaattgagggtcttcaaatc
gtggaaagtgtatttgataatgaacttgttgaagaaataagggagaagat
aaaggacgactttttaggagaatatcgtaaaatttgtgagcttttgtatg
aaaaactcatgaatcgaaagaaaagaggaattgtggttatgactttgggt
tcgagaggggtgcttttctgttcgcacgaaagccctgaagtacaattcct
tccggctattcaaaatgtttcggttgttgatactacaggagctggagata
ctttcctgggcggtttggttactcaattgtatcaaggagagaccttgtct
acggctataaagttctctacattagctagttcattgaccattcaaagaaa
aggtgctgctgaaagcatgccactgtataaagatgttcagaaagatgcat
aa

(2) the RBKSs obtained in the step (1) were ligated into a pET-28a vector to prepare recombinant vectors; and
(3) the prepared recombinant vectors were transformed into an E. coli BL21(DE3) strain respectively to obtain recombinant strains;
the prepared recombinant strains were inoculated into an LB liquid medium respectively, and cultured at 220 rpm and 37° C. for 8-12 hours to prepare seed cultures; and
the prepared seed cultures were inoculated into a TB liquid medium at an inoculum size of 2% (v/v) respectively, and cultured at 220 rpm and 37° C. until OD₆₀₀=approximately 0.6-0.8, isopropyl β-D-thiogalactopyranoside (IPTG) was added to a final concentration of 0.2 mM for induction, and the induction was performed at 220 rpm and 25° C. for 16 hours. After centrifugation, bacterial cells were collected respectively; and
(4) a ribose-5-phosphate (R5P) yield was determined based on a whole-cell reaction of the induced bacterial cells obtained in the step (3).

Reaction conditions: 10 g/L wet bacterial cells prepared were added to a reaction system containing a 10 mL Tris-HCl buffer (50 mM, pH 7.0)(where the reaction system further contained 1 mM DTT, 1 mM EDTA, 5 mM ATP, 5 mM ribose, and 5 mM MgCl₂), and incubated at 37° C. for 10 hours; and after a reaction, a portion of a transformation mixture was taken and centrifuged at 10,000 rpm for 30 minutes, a supernatant was filtered through a 0.22 μm microfiltration membrane, and then measurement by HPLC was performed to determine R5P content in a reaction mixture obtained after the reaction.

Results: a R5P yield of a recombinant strain expressing RBKS derived from E. coli was 3.05 mM;
a R5P yield of a recombinant strain expressing RBKS derived from Saccharomyces cerevisiae was 1.85 mM;
a R5P yield of a recombinant strain expressing RBKS derived from human was 3.97 mM;

### 2. Screening of PRS

(1) PRS derived from E. coli (NCBI No: 945772, SEQ ID NO: 18), PRS derived from Mycobacterium tuberculosis (NCBI No: BAH25337, SEQ ID NO: 19), and PRS derived from Bacillus amyloliquefaciens as shown in SEQ ID NO: 2 were chemically synthesized;

SEQ ID NO: 18:
gtgcctgatatgaagctttttgctggtaacgccaccccggaactagcaca
acgtattgccaaccgcctgtacacttcactcggcgacgccgctgtaggtc
gctttagcgatggcgaagtcagcgtacaaattaatgaaaatgtacgcggt
ggtgatattttcatcatccagtccacttgtgcccctactaacgacaacct
gatggaattagtcgttatggttgatgccctgcgtcgtgcttccgcaggtc
gtatcaccgctgttatcccctactttggctatgcgcgccaggaccgtcgc
gtccgttccgctcgtgtaccaatcactgcgaaagtggttgcagacttcct
ctccagcgtcggtgttgaccgtgtgctgacagtggatctgcacgctgaac
agattcagggtttcttcgacgttccggttgataacgtatttggtagcccg
atcctgctggaagacatgctgcagctgaatctggataacccaattgtggt
ttctccggacatcggcggcgttgtgcgtgcccgcgctatcgctaagctgc
tgaacgataccgatatggcaatcatcgacaaacgtcgtccgcgtgcgaac
gtttcacaggtgatgcatatcatcggtgacgttgcaggtcgtgactgcgt
actggtcgatgatatgatcgacactggcggtacgctgtgtaaagctgctg
aagctctgaaagaacgtggtgctaaacgtgtatttgcgtacgcgactcac
ccgatcttctctggcaacgcggcgaacaacctgcgtaactctgtaattga
tgaagtcgttgtctgcgataccattccgctgagcgatgaaatcaaatcac
tgccgaacgtgcgtactctgaccctgtcaggtatgctggccgaagcgatt
cgtcgtatcagcaacgaagaatcgatctctgccatgttcgaacactaa
SEQ ID NO: 19:
ttgagccacgactggaccgataatcgcaaaaacctgatgctctttgccgg
ccgcgcccatccggagctggccgagcaggtagccaaagagctcgacgtcc
acgtcacctctcaggacgcgcgggagttcgccaacggcgagatcttcgtg
cgcttccacgaatcggtacgcggttgcgacgccttcgtcctgcaatcctg
cccggcaccggtgaacaggtggctgatggaacagctgatcatgatcgacg
cgctcaaacggggcagcgccaaacggatcaccgccgtcatgccgttctat
ccgtatgcccggcaagacaagaaacaccgtggccgcgaaccgatctccgc
gcgactgatcgccgacctgctcaagaccgcgggcgccgaccggatcgtga
cggtcgacctgcacaccgaccagatccagggtttcttcgacgggccggtc
gatcatatgcgcggtcagaacctgctgaccggttacatcagagacaacta
cccggacggcaacatggtggtcgtctcccctgactccggccgggtacgca
tcgccgagaagtgggccgacgcattaggtggtgttcccctcgccttcatc
cacaagacccgtgatccgcgggtacccaaccaagtggtgtccaaccgcgt
cgtcggcgacgtggccgggcgcacctgtgtcctgattgacgacatgatcg
acaccggcggcaccatcgccggcgcggtggcattgctgcacaacgacggc
gccggtgacgtgatcatcgcggcaacccacggcgtgctctccgaccccgc
tgcgcagcggctggcctcctgcggcgcccgcgaagtgatcgtcacgaaca
cgctcccgatcggcgaagacaagcgcttcccccagctcaccgttttgtcc
atcgcgccgctgctggccagcacaatccgggcggtcttcgaaaacggctc
agtaacaggacttttcgacggggacgcatag

(2) the PRSs derived from different sources obtained in the step (1) and the human-derived RBKS as shown in SEQ ID NO: 1 were ligated into a pET-28a vector to prepare recombinant vectors;
(3) the prepared recombinant vectors were transformed into an E. coli BL21(DE3) strain respectively to obtain recombinant strains;
the prepared recombinant strains were inoculated into an LB liquid medium respectively, and cultured at 220 rpm and 37° C. for 8-12 hours to prepare seed cultures; and
the prepared seed cultures were inoculated into a TB liquid medium at an inoculum size of 2% (v/v) respectively, and cultured at 220 rpm and 37° C. until OD₆₀₀=approximately 0.6-0.8, isopropyl β-D-thiogalactopyranoside (IPTG) was added to a final concentration of 0.2 mM for induction, and the induction was performed at 220 rpm and 25° C. for 16 hours. After centrifugation, bacterial cells were collected respectively; and
(4) a PRPP yield was determined based on a whole-cell reaction of the induced bacterial cells obtained in the step (3);
reaction conditions: 10 g/L wet bacterial cells prepared were added to a 10 mL reaction system containing a Tris-HCl buffer (50 mM, pH 7.0) (where the reaction system further contained 1 mM DTT, 1 mM EDTA, 5 mM ATP, 5 mM ribose, and 5 mM MgCl₂), and incubated at 37° C. for 10 hours; and after a reaction, a portion of a transformation mixture was taken and centrifuged at 10,000 rpm for 30 minutes, a supernatant was filtered through a 0.22 μm microfiltration membrane, and then measurement by HPLC was performed to determine PRPP content in a reaction mixture obtained after the reaction.

Results: a PRPP yield of a recombinant strain expressing PRS derived from E. coli was 2.80 mM;
a PRPP yield of a recombinant strain expressing PRS derived from Mycobacterium tuberculosis was 2.92 mM;
a PRPP yield of a recombinant strain expressing PRS derived from Bacillus amyloliquefaciens was 3.35 mM;

### 3. Screening of APRT

(1) APRT derived from Arabidopsis thaliana (NCBI No: 839636, SEQ ID NO: 20), APRT derived from E. coli (NCBI No: 945113, SEQ ID NO: 21), and APRT derived from Schizosaccharomyces pombe as shown in SEQ ID NO: 3 were chemically synthesized;

SEQ ID NO: 20:
tatatatctctaaatgtaaccataacattgtggataataatctgttggag
tgtgaacactctgaaagattctaaatcatccaattttatgtgaagaaata
tgtagaaaaaacaaacaatggatgagttataggtttggttaagacttgaa
aagtcattgatcacggcttttgtccattattccgtgtttaaagtaattta
cattctcttttaagatctctcagtccaaagacaatgcaaacaataataat
ttctccacttgtttctcatcgtctctgtcttgctcgtgctgttccttgca
accgtcttctcaacaaccaccaccgtgctcctccttcgatccgcctctca
aaccaccgttcaaccacctcactccgcctcttctcctccgccggtagacc
ccaccaccttctatttcccttttaaactttttgtttccttttgaaaaaga
tattttgcttgcgtggggcaattccgtcaaagcgttaccattcgggataa
attggtcattttttcatcactgtgtctctctattgagtctttagggtctc
tctgtgtttgcaaatctgacgccgagtattttgctaaagcagcagccagt
cgggacagtgaaatggcgactgaagatgtgcaagatcccagaatcgctaa
gattgcctcttccattagagtcatccccgacttccctaaaccaggtctcc
attttttccatttataccgattaatatcgtctctagtcttgatctgcgtt
gggatcttaaggttttaatttggatacttggcaggaatcatgtttcagga
cataacgacgcttcttctcgacactgaggcctttaaggatactattgctt
tgtttgttgatagatacaaagataaaggcatatctgttgttgcaggtaac
aactcgtatggttctgtttggtactccaatcgattaacagcttgtggatt
tagtttcttttggcctttttagatactgagtgctttagcttaaactgaaa
agttgaaatgaattggtggatgatttttggttctgcctaaattgatgttg
tggtaaatgcaggtgttgaagctagaggtttcatttttggccctcctatt
gcgttggctattggtgccaaatttgttcccatgaggaagcccaagaagct
acctggtacatttttatttcttcctctagttgcttgtgattttgacatta
agttacatgaaaggtctcaattaggaagttgttagtttaaatggcttctt
gctccaagtctacactttatcattatctcgaacattagcaaggtgacttt
tatcttgcagtgtctttgatcaatatgatgctactcatttgctagatcat
ttttctttgtggtaacttgcagtaacttaagtggttgttattttttctgt
tacatatcaatggacttagttgtgtatcttgaccagttacaagaaatgta
ttaagccatttggaaacttatgagcagtgtcaaataattgtcgcattcct
tgtttatccatggacgttaatttctaaactgcctctgcatttggttcctt
taacagggaaggttatttcggaggagtattcgttggagtatggaacagat
acgattgagatgcacgtaggtgcagtagagcctggtgagcgtgctattat
tattgatgacctcattgccacggggggactctcgctgctgcaatccgact
acttggtaagtccaagatttgataatgcagcatatgcagttcagttaaac
ttgactatttctcactcccacaattggttggttttgcagaacgagtagga
gtgaagattgttgaatgtgcttgcgtaattgagttaccagagcttaaggt
ttagcttatttatctctgttttactatctgcttccaatctcttacactga
tttgcttataaagagatatgtgggctgcaatggttttgtagggaaaggag
aaactaggagagacgtcgctatttgttcttgtaaagtcggctgcttaaca
agaaactggaagagaaggttattggatcgagtgttgatgctatttttcat
gtatggtgagacattttgcgtgggatttgatccttgttgtttcaacttat
cataattggttcagactagaaaatggcatttgaatgtcaggattcgattg
cagtttcctattgttccacttaaaatccccgattaataaggtgttatggg
ttttatttatagaatcaagttgtaatagtagactccaacaggggctaaag
tcttggtaacgcaattcttcttttaagtttcgcagcaccttgctcgttac
ttctcttctcaactcgtaaggacacaaatcac
SEQ ID NO: 21:
atgaccgcgactgcacagcagcttgagtatctcaaaaatagcatcaaaag
cattcaggactacccaaaacccggcattcttttccgcgatgtcaccagct
tactggaagacccgaaagcttacgctctcagcatcgacttgctggttgag
cgttacaaaaatgcgggcattaccaaagttgtcggcaccgaagcgcgtgg
cttcttgtttggcgctccggtagctctgggtctgggcgttggctttgtac
cggtccgtaaaccgggcaaactgccgcgtgaaaccatcagtgaaacttac
gacctggaatacggcaccgatcagctggagatccacgttgatgccatcaa
accgggcgacaaagttctggtggtggacgacctgctggcaaccggcggca
ctatcgaagcgaccgttaaactgatccgtcgtctgggtggtgaagtggct
gacgctgcgttcattatcaacctgttcgatctcggcggcgaacagcgtct
cgaaaaacagggcattaccagctacagccttgtcccgttcccgggccatt
aa

(2) the human-derived RBKS as shown in SEQ ID NO: 1, the PRS derived from Bacillus amyloliquefaciens and as shown in SEQ ID NO: 2, and different APRTs derived from different sources obtained in the step (1) were ligated into a pET-28a vector to prepare recombinant vectors;
(3) the prepared recombinant vectors were transformed into an E. coli BL21(DE3) strain respectively to obtain recombinant strains;
the prepared recombinant strains were inoculated into an LB liquid medium respectively, and cultured at 220 rpm and 37° C. for 8-12 hours to prepare seed cultures; and
the prepared seed cultures were inoculated into a TB liquid medium at an inoculum size of 2% (v/v) respectively, and cultured at 220 rpm and 37° C. until OD₆₀₀=approximately 0.6-0.8, isopropyl β-D-thiogalactopyranoside (IPTG) was added to a final concentration of 0.2 mM for induction, and the induction was performed at 220 rpm and 25° C. for 16 hours. After centrifugation, bacterial cells were collected respectively; and
(4) an adenosine monophosphate (AMP) yield was determined based on a whole-cell reaction of the induced bacterial cells obtained in the step (3);
reaction conditions: 10 g/L wet bacterial cells prepared were added to a 10 mL reaction system containing a Tris-HCl buffer (50 mM, pH 7.0) (where the reaction system further contained 1 mM DTT, 1 mM EDTA, 5 mM ATP, 5 mM ribose, 5 mM MgCl₂, and 5 mM adenosine), and incubated at 37° C. for 10 hours; and after a reaction, a portion of a transformation mixture was taken and centrifuged at 10,000 rpm for 30 minutes, a supernatant was filtered through a 0.22 μm microfiltration membrane, and then measurement by HPLC was performed to determine AMP content in a reaction mixture obtained after the reaction.

Results: an AMP yield of a recombinant strain expressing APRT derived from Arabidopsis thaliana was 0.33 mM;
an AMP yield of a recombinant strain expressing APRT derived from E. coli was 1.11 mM;
an AMP yield of a recombinant strain expressing APRT derived from Schizosaccharomyces pombe was 1.76 mM;

### 4. Screening of APSK

(1) APSK derived from E. coli (NCBI No: 947221, SEQ ID NO: 22), APSK derived from Saccharomyces cerevisiae (NCBI No: 853869, SEQ ID NO: 23), and APSK derived from Penicillium chrysogenum as shown in SEQ ID NO: 5 were chemically synthesized;

SEQ ID NO: 22:
atggcgctgcatgacgaaaacgtcgtctggcatagccatccggtcactgt
gcaacaacgcgagctacaccacggtcatcgtggtgtagtgctgtggttta
ccggcctctccgggtccggtaaatcaacggtcgccggggcgctggaggag
gcgttacataaactcggcgtcagtacgtatctgctggatggcgacaatgt
tcgccacggattatgcagcgatctcggttttagcgatgccgatcgtaaag
agaatatccgtcgcgtcggtgaagtggcgaatttgatggttgaagccgga
ctggtggtgctgaccgcatttatctcgccacaccgcgccgaacgccagat
ggttcgcgaacgcgtaggagaagggcgctttatcgaagtgtttgtcgata
cgccgctggcgatttgcgaagcccgcgatcccaaaggcttatataagaaa
gcgcgtgccggtgaactgcgcaactttacgggaatagattccgtttacga
agcgcctgaatcggcagaaattcatctcaatggtgaacaattagtaacaa
atttggtacagcaattattagatctgttgagacagaacgatattatcaga
tcctga
SEQ ID NO: 23:
atggctactaatattacttggcatccaaatcttacttacgacgaacgcaa
ggcattgagaaaacaggacggttgtactatttggttaacaggtctaagtg
cgtcaggtaaaagtacaatcgcctgtgcgctagaacagttactgctccaa
aaaaacttgtctgcatatagattggatggtgacaacattcgttttggatt
gaacaaggatttgggtttctcagaaaaggacagaaatgaaaacattcgta
gaattagcgaagtttctaagctatttgctgattcatgtgctatttcaatc
acctcatttatctctccatacagagttgacagagatagagctcgtgaact
acataaggaggctggtttgaagttcattgaaatatttgttgatgttccat
tagaagtcgctgagcaaagggaccctaagggtttatacaagaaagctagg
gagggtgtaatcaaggagtttacaggtatttctgccccatatgaagcgcc
aaaagctccagagctacatttgagaaccgaccagaagacggttgaagaat
gtgctaccattatttatgagtacttaatcagtgaaaaaatcatccgtaag
catttgtaa

(2) the bovine-derived APSST* as shown in SEQ ID NO: 7 and APSKs derived from different sources obtained in the step (1) were ligated into a pET-28a vector to prepare recombinant vectors;
(3) the prepared recombinant vectors were transformed into an E. coli BL21(DE3) strain respectively to obtain recombinant strains;
the prepared recombinant strains were inoculated into an LB liquid medium respectively, and cultured at 220 rpm and 37° C. for 8-12 hours to prepare seed cultures; and
the prepared seed cultures were inoculated into a TB liquid medium at an inoculum size of 2% (v/v) respectively, and cultured at 220 rpm and 37° C. until OD₆₀₀=approximately 0.6-0.8, isopropyl β-D-thiogalactopyranoside (IPTG) was added to a final concentration of 0.2 mM for induction, and the induction was performed at 220 rpm and 25° C. for 16 hours. After centrifugation, bacterial cells were collected respectively; and
(4) a PAPS yield was determined based on a whole-cell reaction of the induced bacterial cells obtained in the step (3);
reaction conditions: 10 g/L wet bacterial cells prepared were added to a 10 mL reaction system containing a Tris-HCl buffer (50 mM, pH 7.0) (where the reaction system further contained 20 mM AMP, 200 mM Na₂SO₄, 4 mM ATP, and 20 mM MgCl₂), and incubated at 37° C. for 10 hours; and after a reaction, a portion of a transformation mixture was taken and centrifuged at 10,000 rpm for 30 minutes, a supernatant was filtered through a 0.22 μm microfiltration membrane, and then measurement by HPLC was performed to determine PAPS content in a reaction mixture obtained after the reaction.

Results: a PAPS yield of a recombinant strain expressing APSK derived from E. coli was 10.28 mM;
a PAPS yield of a recombinant strain expressing APSK derived from Saccharomyces cerevisiae was 9.11 mM;
a PAPS yield of a recombinant strain expressing APSK derived from Penicillium chrysogenum was 14.12 mM;

### 5. Screening of PPK2

(1) PPK2 derived from E. coli (NCBI No: 946971, SEQ ID NO: 24) and the Rhodobacter sphaeroides-derived PPK2 as shown in SEQ ID NO: 6 were chemically synthesized;

SEQ ID NO: 24:
atgggtcaggaaaagctatacatcgaaaaagagctcagttggttatcgtt
caatgaacgcgtgcttcaggaagcggcggacaaatctaacccgctgattg
aaaggatgcgtttcctggggatctattccaataaccttgatgagttctat
aaagtccgcttcgctgaactgaagcgacgcatcattattagcgaagaaca
aggctccaactctcattcccgccatttactgggcaaaattcagtcccggg
tgctgaaagccgatcaggaattcgacggcctctacaacgagctattgctg
gagatggcgcgcaaccagatcttcctgattaatgaacgccagctctccgt
caatcaacaaaactggctgcgtcattattttaagcagtatctgcgtcagc
acattacgccgattttaatcaatcctgacactgacttagtgcagttcctg
aaagatgattacacctatctggcggtggaaattatccgtggcgataccat
ccgttacgcgctgctggagatcccatcagataaagtgccgcgctttgtga
atttaccgccagaagcgccgcgtcgacgcaagccgatgattcttctggat
aacattctgcgttactgccttgatgatattttcaaaggcttctttgatta
tgacgcgctgaatgcctattcaatgaagatgacccgcgatgccgaatacg
atttagtgcatgagatggaagccagcctgatggagttgatgtcttccagt
ctcaagcagcgtttaactgctgagccggtgcgttttgtttatcagcgcga
tatgcccaatgcgctggttgaagtgttacgcgaaaaactgactatttccc
gctacgactccatcgtccccggcggtcgttatcataattttaaagacttt
attaatttccccaatgtcggcaaagccaatctggtgaacaaaccactgcc
gcgtttacgccatatttggtttgataaagcccagttccgcaatggttttg
atgccattcgcgaacgcgatgtgttgctctattatccttatcacaccttt
gagcatgtgctggaactgctgcgtcaggcttcgttcgacccgagcgtact
ggcgattaaaattaacatttaccgcgtggcgaaagattcacgcatcatcg
actcgatgatccacgccgcacataacggtaagaaagtcaccgtggtggtt
gagttacaggcgcgtttcgacgaagaagccaacattcactgggcgaagcg
cctgaccgaagcaggcgtgcacgttatcttctctgcgccggggctgaaaa
ttcacgccaaactgttcctgatttcacgtaaagaaaacggtgaagtggtg
cgttatgcacacatcgggaccgggaactttaacgaaaaaaccgcgcgtct
ttatactgactattcgttgctgaccgccgatgcgcgcatcaccaacgaag
tacgggggtatttaactttattgaaaacccataccgtccggtgacatttg
attatttaatggtatcgccgcaaaactcccgccgcctattgtatgaaatg
gtggaccgcgagatcgccaacgcgcagcaagggctgcccagtggtatcac
cctgaagctaaataaccttgtcgataaaggcctggttgatcgtctgtatg
cggcctccagctccggcgtaccggttaatctgctggttcgcggaatgtgt
tcgctgatccccaatctggaaggcattagcgacaacattcgtgccatcag
tattgttgaccgttaccttgaacatgaccgggtttatatttttgaaaatg
gcggcgataaaaaggtctacctttcttccgccgactggatgacgcgcaat
attgattatcgtattgaagtggcgacgccgctgctcgatccgcgcctgaa
gcagcgggtactggacatcatcgacatattgttcagcgatacggtcaaag
cacgttatatcgataaagaactcagtaatcgctacgttccccgcggcaat
cgccgcaaagtacgggcgcagttggcgatttatgactacatcaaatcact
cgaacaacctgaataa

(2) the PPK2s derived from different sources obtained in the step (1) were ligated into a vector respectively to prepare recombinant vectors;
(3) the prepared recombinant vectors were transformed into an E. coli BL21(DE3) strain respectively to obtain recombinant strains;
the prepared recombinant strains were inoculated into an LB liquid medium respectively, and cultured at 220 rpm and 37° C. for 8-12 hours to prepare seed cultures; and
the prepared seed cultures were inoculated into a TB liquid medium at an inoculum size of 2% (v/v) respectively, and cultured at 220 rpm and 37° C. until OD₆₀₀=approximately 0.6-0.8, isopropyl β-D-thiogalactopyranoside (IPTG) was added to a final concentration of 0.2 mM for induction, and the induction was performed at 220 rpm and 25° C. for 16 hours. After centrifugation, bacterial cells were collected respectively; and
(4) an ATP yield was determined based on a whole-cell reaction of the induced bacterial cells obtained in the step (3);
reaction conditions: 10 g/L wet bacterial cells prepared were added to a 10 mL reaction system containing a Tris-HCl buffer (50 mM, pH 7.0) (where the reaction system further contained 20 mM ADP, 20 mM sodium hexametaphosphate, and 20 mM MgCl₂), and incubated at 37° C. for 10 hours; and after a reaction, a portion of a transformation mixture was taken and centrifuged at 10,000 rpm for 30 minutes, a supernatant was filtered through a 0.22 μm microfiltration membrane, and then measurement by HPLC was performed to determine ATP content in a reaction mixture obtained after the reaction.

Results: an ATP yield of a recombinant strain expressing PPK2 derived from E. coli was 12.38 mM; and
an ATP yield of a recombinant strain expressing Rhodobacter sphaeroides-derived PPK2 was 18.21 mM.

### Example 2: In Vitro Construction of a Novel PAPS Biosynthesis System

(1) Vectors expressing system enzymes

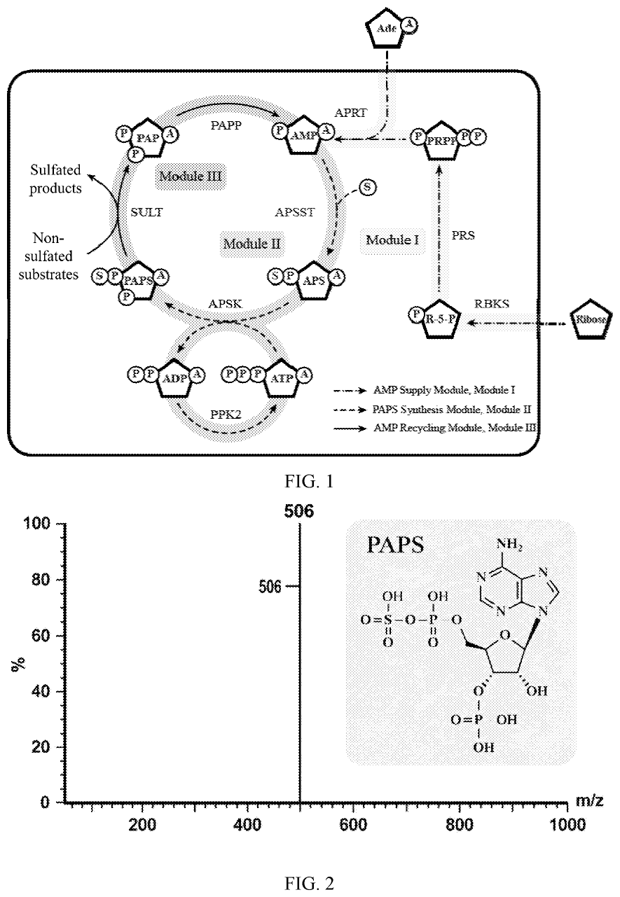

Coding genes of RBKS, PRS, APRT, APSST*, APSK, and PPK2 enzymes as shown in SEQ ID NO: 1, SEQ ID NO: 2, SEQ ID NO: 3, SEQ ID NO: 7, SEQ ID NO: 5, and SEQ ID NO: 6 were chemically synthesized respectively, and ligated into a pET28a vector to obtain recombinant vectors: pET28a-RBKS, pET28a-PRS, pET28a-APRT, pET28a-APSST*, pET28a-APSK, and pET28a-PPK2;
(2) the prepared recombinant vectors were respectively transformed into E. coli BL21 cells, and after induced expression and Ni-column affinity purification, six pure enzymes were obtained;
the six pure enzymes (RBKS, PRS, APRT, APSST*, APSK, and PPK2 were added at amounts of 0.01 mM, 0.02 mM, 0.06 mM, 0.1 mM, 0.1 mM, and 0.1 mM, respectively), ribose (added at an amount of 5 mM), and adenosine (added at an amount of 5 mM) were mixed, a 10 mL Tris-HCl buffer (50 mM, pH 7.5) was added to construct a multi-enzyme cascade system, i.e., an in vitro PAPS biosynthesis system (FIG. 1), a reaction was performed at 30° C. and 220 rpm for 20 minutes, and then the reaction was immediately stopped at 4° C. to obtain a reaction mixture; and
(3) the resulting reaction mixture was centrifuged at 12000 rpm for 15 minutes, a supernatant was freeze-thawed twice at −80° C., a 50 μL sample was diluted 5-fold and immediately centrifuged at 4° C. and 12000 rpm for 15 minutes, and the supernatant was detected by ultra performance liquid chromatography-mass spectrometry (UPLC-MS), where PAPS was successfully detected (FIG. 2), and a molar conversion rate from adenosine to PAPS reached 22.85%, confirming the feasibility of the system.

### Example 3: Construction of PAPS-Producing Escherichia coli Engineered Strain

Specific steps are as follows:
(1) Gene editing of Escherichia coli BL21(DE3) was performed by using a CRISPR-Cas9 system.

Construction of an APSST* integration cassette: Primers cysH-F1, cysH-R1, cysH-F2, and cysH-R2 (Table 2) were designed based on a cysH gene (SEQ ID NO: 8) encoding a PAPS-degrading enzyme (NP_417242.1), and upstream and downstream homology arms of the cysH gene were amplified with a genome of E. coli BL21(DE3) as a template, and cysH-F1/cysH-R1 and cysH-F2/cysH-R2 as primers respectively. A PCR system: 1 μL of 50 ng/μL genomic DNA, 1 μL each of 10 μM primers cysH-F1 and cysH-R1, 25 μL of 2×PrimeSTAR MAX Premix, and 2 μL of ddH₂O₂. PCR conditions: pre-denaturation was performed at 98° C. for 5 minutes; thermal cycling was performed at 98° C. for 10 seconds, 57° C. for 15 seconds, and 72° C. for 1 minute, with 30 cycles; and terminated at 4° C. PCR products were verified by 1% agarose gel electrophoresis and purified by using a PCR Purification Kit.

Three copies of an APSST* gene fragment were synthesized according to a sequence as shown in SEQ ID NO: 8 (the cysH gene).

With cysH-F1/cysH-R2 as a primer, an upstream fragment of cysH, three copies of the APSST* gene, and a downstream fragment of cysH were assembled by PCR fusion to obtain an APSST* integration cassette inserted into a cysH gene locus.

Construction of a pTargetF plasmid: According to a CRISPR/Cas9 gene editing principle, a specific sgRNA sequence targeting the cysH gene was synthesized by Suzhou Genewiz Biotechnology Co., Ltd. and ligated into a pTargetF plasmid to construct pTargetF-cysH.

Transformation of a pCas9 plasmid: The pCas9 plasmid was electroporated into E. coli BL21(DE3) cells, the cells were spread on LB plates containing 50 mg/L kanamycin, and cultured at 30° C. for 24 hours, positive transformants were screened and verified by sequencing to obtain an E. coli BL21(DE3)-pCas9 strain, the strain was cultured in an LB medium containing 50 μg/mL kanamycin and 0.01 M arabinose to induce the expression of homologous recombinase, and cells were collected to prepare electrocompetent cells.

Gene editing: The APSST* integration cassette and the pTargetF-cysH plasmid were co-electroporated into E. coli BL21(DE3)-pCas9 competent cells, after 1 hour of recovery, the cells were spread on LB double-antibiotic plates containing 50 μg/mL kanamycin and 50 μg/mL spectinomycin hydrochloride, and cultured at 30° C. for 24 hours, and positive transformants were selected and verified by colony PCR.

Elimination of the pTargetF plasmid: The positive transformants were transferred to a LB medium, 0.43 mM IPTG was added to induce the expression of endonuclease on the pCas9 plasmid to cleave pTargetF-cysH, and after 12 hours of culturing, the cells were streaked on LB single-antibiotic plates containing 50 μg/mL kanamycin and LB double-antibiotic plates containing 50 μg/mL kanamycin and 50 μg/mL spectinomycin hydrochloride, respectively, and cultured at 30° C. for 12 hours, where strains with the eliminated pTargetF-cysH plasmid only grew on single-antibiotic plates rather than the double-antibiotic plates.

Three copies of the APSST* gene were integrated at a rph pseudogene locus according to the steps (1)-(5), with relevant primers shown in Table 2.

### A Rph Pseudogene Sequence (SEQ ID NO: 38):

atgcgtccagcaggccgtagcaataatcaggtgcgtcccgttaccctgactcgtaactatacaaaacatgcagaaggctc ggtgctggtcgaatttggcgataccaaagtgttgtgtaccgcctctattgaagaaggcgtgccgcgcttcctgaaaggtcagggccag ggctggatcaccgcagagtacggcatgctgccacgttctacccacacccgtaacgctcgtgaagcggcgaaaggtaagcagggtgg acgcacaatggaaatccagcgtctgatcgcccgtgctcttcgcgcggcagtagatttgaaagcgctgggtgagttcaccattacgctg gactgcgacgtgcttcaggctgatggtggcacgcgtaccgcgtcgattacgggtgcctgcgtggcgctggtagatgcgctacagaag ctggtggaaaacggcaagctgaaaaccaatccgatgaaagggatggtagccgcagtttctgtcggaattgtgaacggcgaagcggtt tgcgatctggaatacgttgaagactctgccgcagagaccgacatgaacgtagtgatgaccgaagacgggcgcatcattgaagtgcag gggacggcagaaggcgagccgttcacccatgaagagctactcatcttgttggctctggcccgaggggaatcgaatccattgtag

After gene editing, the strain was cultured at 42° C. to eliminate the pCas9 plasmid and obtain a recombinant strain E. coli BL21(DE3) (ΔcysH, 3APSST*; Δrph, 3APSST*) with cysH knocked out and six copies of APSST* inserted.

table 2
Primers and sequences

| Insertion | | |
|---|---|---|
| locus | Primer | Sequence (5'->3') |
| cysH | cysH-F1 | AGGCAAGGCAAACAGTGAGGAATCTTAATACGACTCACT |
| | | ATAGGGGAATTGTGAGC (SEQ ID NO: 25) |
| | cysH-R1 | AGGCGTTTAATTTATCCGGCAATATCAAAAAACCCCTCAA |
| | | GACCCGT (SEQ ID NO: 26) |
| | cysH-F2 | TGTCAGCGATGAGCATATCGTGA (SEQ ID NO: 27) |
| | cysH-R2 | AATTCCCCTATAGTGAGTCGTATTAAGATTCCTCACTGTTT |
| | | GCCTTGC (SEQ ID NO: 28) |
| rph | rph-F1 | GCGTCGAGATCCTGCTCCT (SEQ ID NO: 29) |
| | rph-R1 | AGTCGTATTAATTGAAATCTCCGGCTTGAAACAAATGT |
| | | (SEQ ID NO: 30) |
| | rph-F2 | GGGTTTTTTGCGACGCAGAAGGCGGC (SEQ ID NO: 31) |
| | rph-R2 | ATTCGCGAATCGCCGTTCC (SEQ ID NO: 32) |

(2) Construction of a system enzyme expression vector

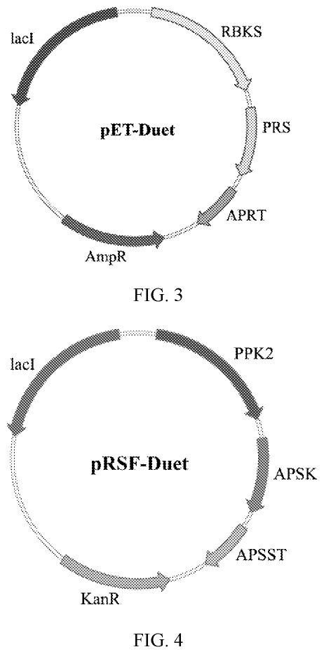

Construction of a pET-Duet-RBKS-PRS-APRT vector: Coding genes of RBKS, PRS, and APRT enzymes were synthesized according to SEQ ID NO: 1-3 and ligated into a pET-Duet vector, such that RBKS, PRS, and APRT were each driven by a T7 promoter (FIG. 3).

A sequence of the T7 promoter is as follows (SEQ ID NO: 37):

taatacgactcactatag

Construction of a pRSF-Duet-APSK-APSST*-PPK2 vector: Coding genes of APSK, APSST*, and PPK2 enzymes were synthesized according to SEQ ID NO: 5, 7, and 6 and ligated into a pRSF-Duet vector, such that APSK, APSST*, and PPK2 were each driven by the T7 promoter (FIG. 4).
(3) Construction of recombinant Escherichia coli

The vectors pET-Duet-RBKS-PRS-APRT and pRSF-Duet-APSK-APSST*-PPK2 were co-electroporated into the recombinant strain E. coli BL21(DE3) (ΔcysH, 3APSST*; Δrph, 3APSST*) constructed in the step (1) to obtain a recombinant strain carrying dual plasmids that was designated as E. coli BL21(DE3) (ΔcysH, 3APSST*; Δrph, 3APSST*)-PAPS, and preserved in glycerol.

### Example 4: Shake Flask Fermentation of Recombinant Escherichia coli

The E. coli BL21(DE3)(ΔcysH, 3APSST*; Δrph, 3APSST*)-PAPS strain constructed in Example 3 was streaked on a plate, and a single colony was picked and inoculated into a LB liquid medium containing 100 μg/mL ampicillin and 50 μg/mL kanamycin, and cultured overnight at 37° C. and 220 rpm. At a 3% (v/v) inoculum size, 5 mL of a seed culture was transferred to a 500 mL shake flask containing 145 mL of a TB medium, and supplemented with 100 μg/mL ampicillin, 50 μg/mL kanamycin, 10 mg/mL adenosine, 10 mg/mL ribose, and 20 mM sodium sulfate.

Fermentation conditions: at an initial temperature 37° C. and a rotation speed of 220 rpm, when OD₆₀₀ reached 0.7, 0.1 mg/mL IPTG was added for induction at a temperature 16° C. and a rotation speed of 200-250 rpm, and fermentation continued for 35 hours. After fermentation, a final volume was measured, cells were collected by centrifugation and disrupted, a cell lysate was collected by centrifugation, and a PAPS yield was determined by HPLC. E. coli BL21(DE3) was used as a control.

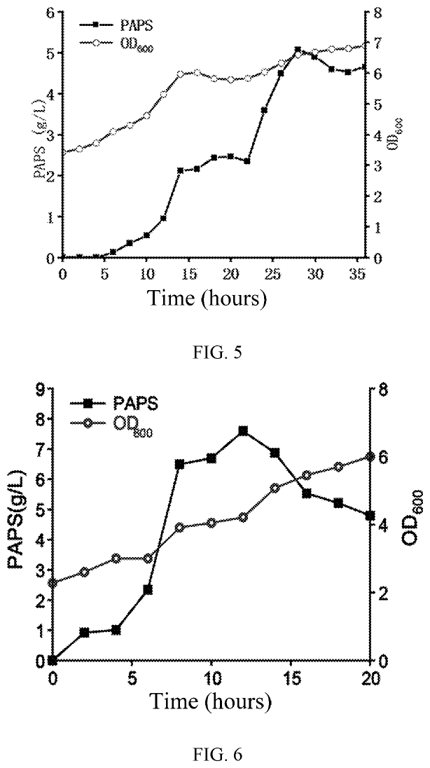

As shown in FIG. 5, a PAPS yield of the recombinant strain E. coli BL21(DE3)(ΔcysH, 3APSST*; Δrph, 3APSST*)-PAPS reached 5.08 g/L after shake flask fermentation.

### Example 5: 5-L Fermenter Fermentation of Recombinant Escherichia coli

Seed culture preparation is the same as that in Example 4. 2.25 L of a fermentation medium was prepared, and 250 mL of a seed culture was injected into a fermenter through an inoculation port at a 10% (v/v) inoculum size, where aseptic conditions were maintained throughout the process, with an initial working volume of 2.5 L. Fermentation conditions: an initial rotation speed of 400 rpm, an initial aeration rate of 1.0 vvm, initial pH 7.0, and a temperature of 37° C.; when OD₆₀₀ reached 2.5, IPTG (0.4 mM), adenosine (10 mg/mL), ribose (10 mg/mL), and sodium sulfate (20 mM) were added through a feed port, fermentation continued for 20 hours, and throughout the fermentation cycle, concentrated ammonia water was automatically added to control the pH at 7.0. After fermentation, a final volume was measured, cells were collected by centrifugation and disrupted, a supernatant of a cell lysate was collected, and a PAPS yield was determined by HPLC. E. coli BL21(DE3) was used as a control.

As shown in FIG. 6, a PAPS yield of the recombinant strain E. coli BL21(DE3)(ΔcysH, 3APSST*; Δrph, 3APSST*)-PAPS reached 7.60 g/L after 5-L fermenter fermentation.

### Example 6: Construction of a PAPS-Producing Engineered Strain of Bacillus subtilis

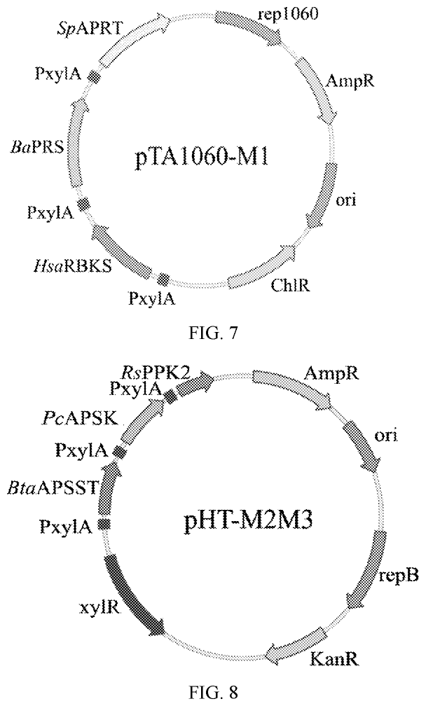

Specific steps were the same as those in Example 3: First, Bacillus subtilis 168 was used as a chassis strain to construct a recombinant strain B. subtilis 168 (ΔcysH, 6APSST*) with cysH knocked out and six copies of APSST inserted, primers used were shown in Table 3, and APSST* was inserted at a cysH gene locus. Further, the pTA-RBKS-PRS-APRT vector (FIG. 7) and the pHT-APSK-APSST-PPK2 vector (FIG. 8) were constructed and co-electroporated into B. subtilis 168 (ΔcysH, 6APSST*) to obtain a recombinant strain carrying dual plasmids that was designated as B. subtilis 168 (ΔcysH, 6APSST*)-PAPS and preserved in glycerol.

TABLE 3
Primers and sequences

| Insertion | | |
|---|---|---|
| locus | Primer | Sequence (5′->3′) |
| cysH | Bs-cysH-F1 | TAATACGACTCACTATAGGGGAATTG |
| | | TGAGC (SEQ ID NO: 33) |
| | Bs-cysH-R1 | TTATTGCTCAGCGGTGGCAG |
| | | (SEQ ID NO: 34) |
| | Bs-cysH-F2 | CTGCCACCGCTGAGCAATAATAATAC |
| | | GACTCACTATAGGGGA |
| | | ATTGTGAGC (SEQ ID NO: 35) |
| | Bs-cysH-R2 | ATCTCAGTGGTGGTGGTGGT |
| | | (SEQ ID NO: 36) |

### Example 7: 5-L Fermenter Fermentation of Recombinant Bacillus subtilis

The B. subtilis 168 (ΔcysH, 6APSST*)-PAPS strain constructed in Example 6 was streaked on a plate, and a single colony was picked and inoculated into a LB liquid medium containing 100 μg/mL chloramphenicol and 50 μg/mL kanamycin, and cultured overnight at 37° C. and 220 rpm. Fermentation conditions: an initial rotation speed of 400 rpm, an initial aeration rate of 1.0 vvm, initial pH 7.0, and a temperature of 37° C.; when OD₆₀₀ reached 12.5, xylose (0.4 mM), adenosine (10 mg/mL), ribose (10 mg/mL), and sodium sulfate (20 mM) were added through a feed port, fermentation continued for 20 hours, and throughout the fermentation cycle, concentrated ammonia water was automatically added to control the pH at 7.0. After fermentation, a final volume was measured, cells were collected by centrifugation and disrupted, a cell lysate was collected by centrifugation, and a PAPS yield was determined by HPLC. B. subtilis 168 was used as a control.

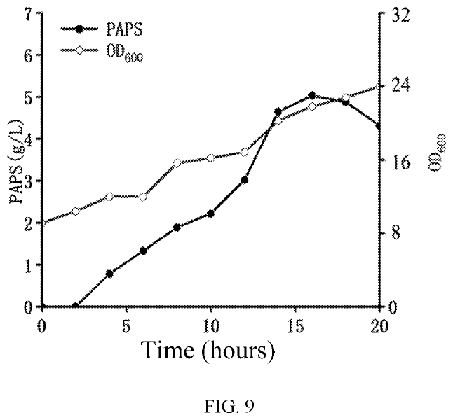

As shown in FIG. 9, a PAPS yield of the recombinant strain B. subtilis 168 (ΔcysH, 6APSST*)-PAPS reached 5.03 g/L after 5-L fermenter fermentation.

Although the present disclosure has been disclosed in preferred examples, but they are not intended to limit the present disclosure. Anyone skilled in the art can make various changes and modifications without departing from the spirit and scope of the present disclosure. Therefore, the scope of protection of the present disclosure should be defined by the claims.

What is claimed is:

1. A novel biosynthesis system for producing 3′-phosphoadenosine-5′-phosphosulfate, comprising the following enzymes: ribokinase (RBKS), phosphoribosyl pyrophosphate synthetase (PRS), adenine phosphoribosyltransferase (APRT), adenine sulfotransferase (APSST), adenosine 5′-phosphosulfate kinase (APSK), and polyphosphate kinase 2 (PPK2).

2. The biosynthesis system according to claim 1, wherein the amino acid sequence of RBKS is set forth in SEQ ID NO:9; the amino acid sequence of PRS is set forth in SEQ ID NO:10; the amino acid sequence of APRT is set forth in SEQ ID NO:11; the amino acid sequence of APSST is set forth in SEQ ID NO:12 or SEQ ID NO:15; the amino acid sequence of APSK is set forth in SEQ ID NO:13; and the amino acid sequence of PPK2 is set forth in SEQ ID NO:14.

3. The biosynthesis system according to claim 2, wherein the nucleotide sequence of a gene encoding RBKS is set forth in SEQ ID NO:1; the nucleotide sequence of a gene encoding PRS is set forth in SEQ ID NO:2; the nucleotide sequence of a gene encoding APRT is set forth in SEQ ID NO:3; the nucleotide sequence of a gene encoding APSST is set forth in SEQ ID NO:4 or SEQ ID NO:7; the nucleotide sequence of a gene encoding APSK is set forth in SEQ ID NO:5; and the nucleotide sequence of a gene encoding PPK2 is set forth in SEQ ID NO:6.

4. An application of the biosynthesis system according to claim 1 in producing 3′-phosphoadenosine-5′-phosphosulfate, wherein specifically 3′-phosphoadenosine-5′-phosphosulfate is obtained by catalyzing a reaction where enzymes in the biosynthesis system are used as catalysts, and ribose and adenosine serve as substrates.

5. The application according to claim 4, wherein amounts of enzymes added in the biosynthesis system are as follows: an amount of RBKS added is 0.005-0.015 mM, an amount of PRS added is 0.01-0.03 mM, an amount of APRT added is 0.05-0.07 mM, an amount of APSST added is 0.05-0.15 mM, an amount of APSK added is 0.05-0.15 mM, and an amount of PPK2 added is 0.05-0.15 mM; and an amount of ribose added is 1-10 mM and an amount of adenosine added is 1-10 mM.

6. The application according to claim 4, wherein the catalytic reaction is performed at 25-35° C. and 200-300 rpm for 10-60 minutes.

7. A composition for producing 3′-phosphoadenosine-5′-phosphosulfate, comprising enzymes in the biosynthesis system according to claim 1, wherein the enzymes comprise RBKS, PRS, APRT, APSST, APSK, and PPK2; the amino acid sequence of RBKS is set forth in SEQ ID NO:9; the amino acid sequence of PRS is set forth in SEQ ID NO:10; the amino acid sequence of APRT is set forth in SEQ ID NO:11; the amino acid sequence of APSST is set forth in SEQ ID NO:12 or SEQ ID NO:15; the amino acid sequence of APSK is set forth in SEQ ID NO:13; and the amino acid sequence of PPK2 is set forth in SEQ ID NO:14.

8. The composition according to claim 7, wherein the nucleotide sequence of a gene encoding RBKS is set forth in SEQ ID NO:1; the nucleotide sequence of a gene encoding PRS is set forth in SEQ ID NO:2; the nucleotide sequence of a gene encoding APRT is set forth in SEQ ID NO:3; the nucleotide sequence of a gene encoding APSST is set forth in SEQ ID NO:4 or SEQ ID NO:7; the nucleotide sequence of a gene encoding APSK is set forth in SEQ ID NO:5; and the nucleotide sequence of a gene encoding PPK2 is set forth in SEQ ID NO:6.

9. An engineered strain, wherein the engineered strain expresses RBKS, PRS, APRT, APSST, APSK, and PPK2; the amino acid sequence of RBKS is set forth in SEQ ID NO:9; the amino acid sequence of PRS is set forth in SEQ ID NO:10; the amino acid sequence of APRT is set forth in SEQ ID NO:11; the amino acid sequence of APSST is set forth in SEQ ID NO:12 or SEQ ID NO:15; the amino acid sequence of APSK is set forth in SEQ ID NO:13; and the amino acid sequence of PPK2 is set forth in SEQ ID NO:14.

10. The engineered strain according to claim 9, wherein the nucleotide sequence of a gene encoding RBKS is set forth in SEQ ID NO:1; the nucleotide sequence of a gene encoding PRS is set forth in SEQ ID NO:2; the nucleotide sequence of a gene encoding APRT is set forth in SEQ ID NO:3; the nucleotide sequence of a gene encoding APSST is set forth in SEQ ID NO:4 or SEQ ID NO:7; the nucleotide sequence of a gene encoding APSK is set forth in SEQ ID NO:5; and the nucleotide sequence of a gene encoding PPK2 is set forth in SEQ ID NO:6.

11. The engineered strain according to claim 10, wherein a host cell of engineered strain is Escherichia coli or Bacillus subtilis.

12. The engineered strain according to claim 11, wherein when a host of the engineered strain is Escherichia coli, the engineered strain further comprises three copies of a gene encoding APSST set forth in SEQ ID NO:7 inserted at a cysH gene locus of a genome, and three copies of the gene encoding APSST set forth in SEQ ID NO:7 inserted at a rph pseudogene locus; and when the host of the engineered strain is Bacillus subtilis, the engineered strain further comprises six copies of the gene encoding APSST set forth in SEQ ID NO:7 inserted at the cysH gene locus of the genome.

13. The engineered strain according to claim 11, wherein the engineered strain, using Escherichia coli as a host cell, expresses RBKS, PRS, and APRT by a pET-Duet expression vector, and expresses APSST, APSK, and PPK2 by a pRSF-Duet expression vector; RBKS, PRS, APRT, APSST, APSK, and PPK2 are all expressed under control of a T7 promoter; three copies of a gene encoding APSST set forth in SEQ ID NO:7 are inserted into a cysH gene locus having the nucleotide sequence set forth in SEQ ID NO:8 in a genome, and three copies of a gene encoding APSST set forth in SEQ ID NO:7 are inserted into a rph pseudogene locus with the nucleotide sequence set forth in SEQ ID NO:38; the nucleotide sequence of a gene encoding RBKS is set forth in SEQ ID NO:1; the nucleotide sequence of a gene encoding PRS is set forth in SEQ ID NO:2; the nucleotide sequence of a gene encoding APRT is set forth in SEQ ID NO:3; the nucleotide sequence of a gene encoding APSST is set forth in SEQ ID NO:7; the nucleotide sequence of a gene encoding APSK is set forth in SEQ ID NO:5; the nucleotide sequence of a gene encoding PPK2 is set forth in SEQ ID NO:6; and the engineered strain is designated as strain E. coli BL21(DE3) (ΔcysH, 3APSST*; Δrph, 3APSST*)-PAPS.

14. A recombinant host cell comprising nucleic acids encoding enzymes in the biosynthesis system according to claim 1, wherein the enzymes comprise RBKS, PRS, APRT, APSST, APSK, and PPK2; the nucleotide sequence of a gene encoding RBKS is set forth in SEQ ID NO:1; the nucleotide sequence of a gene encoding PRS is set forth in SEQ ID NO:2; the nucleotide sequence of a gene encoding APRT is set forth in SEQ ID NO:3; the nucleotide sequence of a gene encoding APSST is set forth in SEQ ID NO:4 or SEQ ID NO:7; the nucleotide sequence of a gene encoding APSK is set forth in SEQ ID NO:5; and the nucleotide sequence of a gene encoding PPK2 is set forth in SEQ ID NO:6.

15. An application of the engineered strain according to claim 9 in fermentative production of 3′-phosphoadenosine-5′-phosphosulfate, wherein the application comprises de novo synthesis of 3′-phosphoadenosine-5′-phosphosulfate using ribose and adenosine as substrates with the engineered strain.

16. A method for fermentatively producing 3′-phosphoadenosine-5′-phosphosulfate using the engineered strain according to claim 9, wherein the method comprises synthesizing 3′-phosphoadenosine-5′-phosphosulfate using ribose and adenosine as substrates with the engineered strain.
